(19) United States
(12) Patent Application Publication
(10) Pub. No.: US 2008/0200434 A1
(43) Pub. Date: Aug. 21, 2008

(54) Chemical Target-Binding Compositions

(76) Inventor: George Y. Daniloff, Mountain View, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(21) Appl. No.: 12/047,076

(22) Filed: Mar. 12, 2008

(30) Foreign Application Priority Data
Sep. 14, 2006 (US) PCT/US2006/035968

(51) Int. Cl. A61K 31/69

(52) U.S. Cl. 514/64

(57) ABSTRACT

Herein is described compositions and methods for modulating and regulating the concentration of a chemical target in an organism or in an ecosystem. In one embodiment, the concentration is regulated such that it is maintained below or above a certain threshold, thus avoiding toxic or harmful effects of the chemical target. The described compositions may, for example, be implanted in a human body or placed in the ecosystem. The compositions comprise binding moieties that reversibly bind to a chemical target in the organism or ecosystem. The binding capacity and the binding constants for a chemical target are designed such that the composition maintains the concentration of the chemical target substantially within a beneficial range of concentrations.

### CROSS-REFERENCE TO RELATED APPLICATIONS

This application claims priority to Patent Cooperation Treaty (international) application no. PCT/US2006/035968, whose international filing date is Sep. 14, 2006, and which claims priority under 35 U.S.C. §119 to Provisional U.S. Patent Application Ser. No. 60/596,300, filed Sep. 14, 2005. PCT/US2006/035968 designated the United States and published in English. The disclosures of the priority applications are incorporated by reference herein.

### TECHNICAL FIELD

This disclosure relates generally to compositions useful in regulating the concentration of chemical targets in a fluid. The invention finds utility, for example, in the fields of medicine and environmental science.

### BACKGROUND

Regulation of the concentration of substances in aqueous environments is as important to the health of humans and other organisms as to the health of ecosystems. For example, in the case of humans, regulating the blood concentration of various metabolites to remain within physiologically acceptable limits is key to survival. A prolonged deficiency or surplus of nearly any of the components of blood can produce adverse systemic effects. In addition, acute or short-term variations in blood concentrations can be highly toxic. Similarly, the overall health of environmental ecosystems requires regulation of the aqueous concentration of nutrients and other chemicals within certain limits.

Organisms, as well as ecosystems, are able to tolerate the presence of toxic compounds under certain circumstances. According to the threshold theory of toxicity, a toxic substance must be present in an organism at some threshold concentration before any adverse effects are evident. Below this concentration, no such adverse effects are observed. By using this theory it is assumed that tiny doses of a toxic substance will not cause any adverse effects. A toxic substance must be present in an organism at some “threshold” concentration before any toxic effects are evident.

Therefore, the health of an organism or ecosystem relies on an ability to maintain healthy concentrations of all of the chemical species that are present within the organism or ecosystem. This principle applies to nutrients, toxins, metabolites, and other chemicals regularly encountered in aqueous environments.

In a healthy person, for example, insulin is secreted by the pancreas in response to rising levels of blood glucose, thereby helping to maintain blood glucose concentrations near 5 mM. In type 1 diabetic patients, insulin secretion is impaired or annihilated, and daily injections of insulin are therefore vital. State-of-the-art insulin therapy utilizes short and long acting insulins (meal and basal insulins) in various regimens, thereby attempting to maintain glucose levels in the diabetic patient at a healthy level at all times. Tight glucose control is paramount in avoiding long-term tissue damage (eyes, kidneys, heart, etc.) from hyperglycemia (>10 mM) and acute danger (coma or death) from hypoglycemia (<3 mM). However, in many patients, unexpected/irregular changes in glucose levels occur almost daily, and these events are not easily compensated for with current state-of-the-art insulin therapy. It has been shown that glycemic variability in diabetic patients may be an important factor involved in the pathogenesis of microvascular complications. It is now appreciated that oxidative stress from overproduction of reactive oxygen species may be the result of this glycemic variability (Hirsch I B, Am J. Med. 2005 May; 118(Suppl 5A):21S-6S).

Current methods of regulating blood levels of insulin, for example, involve measuring the concentration in the blood and taking steps to increase or decrease blood insulin levels based on the results of the measurements. However, this method requires active and regular participation by the patient. In the absence of vigilant monitoring, glucose levels may become damagingly unhealthy in a very short amount of time. In addition, the required blood tests and insulin injections place a variety of limitations on the lifestyles of patients. Because of the influence of human activities, many ecosystems also require a variety of tests and preventative or remedial applications of various chemicals in order to remain healthy.

The present invention is directed at addressing one or more of the above-mentioned drawbacks, as well as related issues in the field of medicinal chemistry and environmental science.

### SUMMARY OF THE DISCLOSURE

The present disclosure describes compositions, implants, formulations, medical devices and methods for modulating and controlling the concentration of a chemical target in vivo or in an ecosystem. The described compositions have a capacity to bind the chemical target present in the ecosystem or the physiological fluid of a living organism in a reversible manner. The capacity for binding with the chemical target and the binding constants for the chemical target are designed to maintain the concentration of the chemical target substantially within a beneficial range of concentrations.

In one embodiment, then, the disclosure describes a pharmaceutical composition comprising a biocompatible binding component. The binding component comprises a binding moiety capable of reversibly binding with a chemical target. The pharmaceutical composition is capable of regulating the concentration of the chemical target in a physiological fluid of a patient. A method of regulating the concentration of a chemical target in a physiological fluid of a patient is also described. The method comprises contacting the physiological fluid with such a composition.

In another embodiment, the disclosure describes a pharmaceutical composition for treating diabetes. The pharmaceutical composition comprises a biocompatible binding component capable of reversibly binding glucose. The pharmaceutical composition is in a dosage form capable of contacting the physiological fluid of a patient suffering from diabetes for a predetermined length of time.

In yet another embodiment, the disclosure describes a method for regulating the concentration of glucose in the blood of a patient. The method comprises contacting the blood of the patient with a pharmaceutical composition comprising a therapeutic amount of a biocompatible binding component capable of reversibly binding glucose.

In yet another embodiment, the disclosure describes a composition for regulating the concentration of a chemical target in an aqueous environment of an ecosystem. The composition comprises a binding component. The binding component comprises a binding moiety capable of reversibly binding with a chemical target. The binding constant of the binding moiety is complementary to the beneficial range of concentrations for the chemical target in the aqueous environment.

In another embodiment, the disclosure describes glucose-binding composition and medical device capable of reversible glucose binding in the physiological environment. In addition, this embodiment describes methods of use of the glucose-binding composition and device for the treatment of diabetes, wound healing, tissue regeneration and tissue repair. In yet another embodiment, the chemical target can be any other physiological agent, metabolic byproduct, natural compound or a synthetic chemical entity present in the living body or ecosystem. For example, the chemical target is a drug, pharmaceutical agent, biological or physiological process altering agent found in or used in a living body or ecosystem.

In a further embodiment, the disclosure describes a drug-binding composition that is implanted in an organism. Furthermore, the drug is introduced (separately or together with the drug-binding composition) into the organism by conventional medication routes (injection, ingestion, inhalation, etc.). Drug circulating in the body after such introduction is bound by the implanted drug-binding composition and subsequently released into circulation when the circulating drug concentration is diminished. The specificity, binding constants, capacity and binding and dissociation rates of the drug by the drug-binding moieties can be optimized to achieve desired kinetics of drug delivery.

In one embodiment, a chemical target is an endogenous biologically or physiologically active agent such as hormone, growth factor, cytokine, etc. The endogenous factor is present in the living body or ecosystem. The binding composition in this case is used as “sponge” for the endogenous agent. Such a binding composition may be useful, for example, in concentrating and localizing the effect of the chemical target at the local tissue site where the binding composition is implanted.

In yet another embodiment, the chemical target can be a toxin or pollutant, or other undesirable agent present in living body or ecosystem. The binding composition binds the toxin at the concentration levels that are toxic to the living body or ecosystem and may release the toxin by dissociation when toxin levels fall sufficiently below the toxic concentration levels.

This disclosure also describes methods of treating diabetes and its complications by improving glycemic control and reducing glycemic variations.

### DETAILED DESCRIPTION OF THE PREFERRED EMBODIMENTS

### Definitions and Nomenclature

Before describing the present invention in detail, it is to be understood that unless otherwise indicated, this invention is not limited to particular metabolite-binding moieties, compositional forms, polymers, polymerization techniques, synthetic methods, crosslinking techniques, or methods of use, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, “a glucose-binding moiety” refers not only to a single glucose binding molecular structure or moiety but also to a combination of two or more different moieties, “a hydrophilic polymer” refers to a combination of hydrophilic polymers as well as to a single hydrophilic polymer, and the like. Unless defined otherwise, all technical and scientific terms used herein have the meaning commonly understood by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

When referring to the value of a variable, the value may be described as “substantially within” a given range over time. By this is meant that the value of the variable remains within the given range at least 90%, preferably 95%, of the time that the variable is measured. Thus the term “substantially within” takes allowance for isolated and relatively short-lived deviations of the value of the variable beyond the limits of the given range.

The term “living body” refers to human or animal body or its parts, organs, systems, tissues, cells, cell cultures, and includes body fluids such as blood, lymph, cerebrospinal fluid, saliva, tears, sweat, or urine.

The term “ecosystem” refers to a community of plants, animals, and microorganisms that are linked by energy and nutrient flows and that interact with each other and with the physical environment. Rain forests, ocean, lakes, rivers, ponds, deserts, coral reefs, grasslands, and a rotting log are all examples of ecosystems.

The term “beneficial range of concentration” refers to the concentration of a chemical present in the living body or ecosystem at which said chemical is not harmful if, for example, the chemical is a toxin or pollutant; or at which said chemical is therapeutically effective if, for example, the chemical is a drug; or refers to the normal and/or healthy range of concentrations of the chemical if, for example, the chemical is a nutrient such as glucose. In some cases, a chemical target will have a “beneficial concentration” rather than a beneficial range of concentrations in the physiological fluid or ecosystem. The term “beneficial range of concentrations,” as used herein, is meant to encompass instances wherein the chemical target has a beneficial range of concentrations as well as instances wherein the chemical target has a single beneficial concentration. In general, beneficial ranges of concentrations are known in the art for a wide variety of substances found in or added to physiological fluid or ecosystems.

A binding constant may be described herein as “complementary” to a beneficial range of concentrations. By this is meant that the binding constant of a binding moiety is such that, for a composition containing the binding moiety, the equilibrium concentration of a chemical target in the composition is within the beneficial range of concentrations. The equilibrium concentration of a chemical target is the concentration at which binding and releasing of the chemical target with the binding moieties is in equilibrium.

The term “polymer” is used to refer to molecules composed of repeating monomer units, including homopolymers, block copolymers, random copolymers, and graft copolymers. Any molecule having at least 2 carbon atoms is deemed to be a polymer for the purposes of this invention.

The term “prepolymer” refers to monomers, oligomers and polymers that can be further used to create a larger molecular weight polymer by crosslinking, polymerizing or otherwise linking prepolymers.

The term “crosslinked” herein refers to a composition containing intermolecular crosslinks and, optionally, intramolecular crosslinks as well, arising from the formation of covalent bonds. Covalent bonding between two crosslinkable components may be direct, in which case an atom in one component is directly bound to an atom in the other component, or it may be indirect, through a linking group. A crosslinked matrix may, in addition to covalent bonds, also include intermolecular and/or intramolecular non-covalent bonds and electrostatic (ionic) bonds. The term “crosslinkable” refers to a component or compound that is capable of undergoing reaction to form a crosslinked composition.

The term “synthetic” to refer to various polymers, drugs, toxins or pollutants herein is intended to mean “chemically synthesized.” Therefore, a synthetic polymer in the present composition may have a molecular structure that is identical to a naturally occurring polymer, but the polymer per se, as incorporated in the composition of the invention, has been chemically synthesized in the laboratory or industrially. “Synthetic” polymers also include semi-synthetic polymers, i.e., naturally occurring polymers, obtained from a natural source, that have been chemically modified in some way. Generally, however, the synthetic polymers herein are purely synthetic, i.e., they are neither semi-synthetic nor have a structure that is identical to that of a naturally occurring polymer.

The terms “hydrophilic” and “hydrophobic” are generally defined in terms of a partition coefficient P, which is the ratio of the equilibrium concentration of a compound in an organic phase to that in an aqueous phase. A hydrophilic compound has a log P value of less than 1.0, typically of less than about 0.5, where P is the partition coefficient of the compound between octanol and water, while hydrophobic compounds will generally have a log P greater than about 3.0, typically greater than about 5.0.

Polymers may be crosslinked by either “physical” or chemical means. Physical crosslinking differs from chemical crosslinking in that the linkages are typically weaker, of lower energy, and often reversible. Thus, physically crosslinked hydrogels often are deformable mechanically. Four fundamental forces have been found to be responsible for producing physical crosslinking: ionic interactions; hydrophobic interactions; hydrogen bonding and Van der Waals forces.

“Physical crosslinking” may be intramolecular or intermolecular or in some cases, both. For example, hydrogels can be formed by the ionic interaction of divalent cationic metal ions (such as Ca+2 and Mg+2) with ionic polysaccharides such as alginates, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, and amylopectin. These crosslinks may be easily reversed by exposure to species that chelate the crosslinking metal ions, for example, ethylene diamine tetraacetic acid. Multifunctional cationic polymers, such as poly(1-lysine), poly(allylamine), poly(ethyleneimine), poly(guanidine), poly(vinyl amine), which contain a plurality of amine functionalities along the backbone, may be used to further induce ionic crosslinks.

As used herein, “derivative” refers to a chemically or biologically modified version of a chemical compound that is structurally similar to a parent compound and (actually or theoretically) derivable from that parent compound. A “derivative” differs from an “analog” in that a parent compound may be the starting material to generate a “derivative,” whereas the parent compound may not necessarily be used as the starting material to generate an “analog.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange

As used herein, “analog” refers to a chemical compound that is structurally similar to a parent compound, but differs slightly in composition (e.g., one atom or functional group is different, added, or removed). The analog may or may not have different chemical or physical properties than the original compound and may or may not have improved biological and/or chemical activity. For example, the analog may be more hydrophilic or it may have altered reactivity as compared to the parent compound. The analog may mimic the chemical and/or biologically activity of the parent compound (i.e., it may have similar or identical activity), or, in some cases, may have increased or decreased activity. The analog may be a naturally or non-naturally occurring (e.g., recombinant) variant of the original compound. An example of an analog is a mutein (i.e., a protein analog in which at least one amino acid is deleted, added, or substituted with another amino acid). Other types of analogs include isomers (enantiomers, diasteromers, and the like) and other types of chiral variants of a compound, as well as structural isomers. The analog may be a branched or cyclic variant of a linear compound. For example, a linear compound may have an analog that is branched or otherwise substituted to impart certain desirable properties (e.g., improve hydrophilicity or bioavailability).

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials which may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or protonation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time. Examples of biodegradable synthetic polymers include polyglycolic acid (PGA) and poly-DL-lactide-co-glycolide (PLGA), crosslinked hyaluronic acid, crosslinked PEG containing carboxylic ester or thioester linkages.

The term “small molecular weight crosslinker” refers to multi reactive group containing molecules with molecular weights between approximately 100 and 3,000 Daltons. For example, reagents containing two or more succinimidyl groups are small molecular weight crosslinkers including disuccinimidyl suberate (DSS), bis(sulfosuccinimidyl) suberate (BS₃), dithiobis(succinimidylpropion-ate) (DSP), bis(2-succinimidooxycarbonyloxy) ethyl sulfone (BSocOES), and 3,3′-dithiobis(sulfosuccinimidylpropionate (DTSPP), and their analogs and derivatives. The above-referenced polymers are commercially available from Pierce (Rockford, Ill.).

The term “synthetic hydrophilic polymer” as used herein refers to a synthetic polymer composed of molecular segments that render the polymer as a whole “hydrophilic,” as defined above. Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions. Hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol; polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines including poly(methyloxazoline) and poly(ethyloxazoline); and polyvinylamines.

The term “activated” refers to a modification of an existing functional group to generate or introduce a new reactive functional group from an existing functional group, wherein the new reactive functional group is capable of undergoing reaction with another functional group to form a covalent bond. For example, a component containing carboxylic acid (—COOH) groups can be activated by reaction with N-hydroxy-succinimide or N-hydroxysulfosuccinimide using known procedures, to form an activated carboxylate (which is a reactive electrophilic group), i.e., an N-hydroxysuccinimide ester or an N-hydroxysulfosuccinimide ester, respectively. In another example, carboxylic acid groups can be activated by reaction with an acyl halide such as acyl chloride to provide an activated electrophilic group in the form of an anhydride.

The term “particle surface” refers to the total surface including the outer surface and surface created by porosity, texture of the surface, cracks, channels, or other structures available for contact with the components of physiological tissue fluids or tissue surface. In one aspect of the invention the particle surface is increased by grafting branched hydrophilic polymers with side chains containing multiple reactive groups and glucose binding groups.

As used herein, “biomaterial” refers to compositions suitable for implantation, contact, ingestion, inhalation, or other introduction into the living body, or interface with the living body. The biomaterial can be prepared ex-vivo or formed in-vivo using synthetic or naturally occurring, hydrophilic or hydrophobic, biodegradable or non-biodegradable polymers, prepolymers, monomers, or minerals.

The term “reactive moieties at the tissue site” refers to nucleophilic groups such as primary and secondary amines, sulfhydryls, hydroxyls and other reactive chemical groups found in physiological fluids and on the tissue surface. The reactive moieties available to react with activated groups of the implant can be found on amino acids, peptides, proteins, lipids, proteoglycans, cell surface proteins, extra cellular matrix, cell breakdown components, blood proteins and cells, wound exudates, blood, plasma and lymph.

The term “implant” herein refers to any composition or object placed surgically or otherwise in contact with a human or animal body. Such implants can have a diagnostic, therapeutic, or aesthetic function, or can be used as identification, or information storage or processing devices, and include without limitations all know medical implants and devices. Said implants can be attached on the outside surfaces of the body such as skin, oral mucosa, teeth, nails, eye, and ear-nose-throat passages, or can be placed inside of pulmonary system, or digestive system, or urinary system, or intestinal tract, or reproductive system, or vascular system, or surgically placed subcutaneously, intramuscularly, intraperitoneally, or in any other location in the body.

As used herein, unless otherwise specified, the term “device” refers to any form of the compositions described herein that may be suitable for delivery to an organism or ecosystem. The term therefore encompasses embodiments wherein the compositions are formulated as a medical dosage form (e.g., pills, implants, pastes, solutions, etc.) as well as wherein the compositions are formulated in an environmental delivery device (e.g., sponges, particles, solutions, etc.).

The term “physiological fluid,” as used herein, refers to any fluid normally found within an organism such as a human or other animal. Physiological fluids include blood and the various components of blood, cerebral spinal fluid, synovial fluid, saliva, gastrointestinal fluids, urine, semen, etc.

### Compositions

### Chemical Target

The “chemical target,” or simply “target,” refers to a compound, composition, molecule, element, ion, biomolecule, or fragment thereof that may be found in or added to a living body or ecosystem. Chemical targets are not limited to naturally occurring substances. The compositions disclosed herein may contain the chemical target. Chemical targets include metabolites, nutrients, metabolic breakdown products, toxins, drugs, pharmaceuticals, biopharmaceuticals, drugs of abuse, complexing agents, hormones, detergents, fertilizers, herbicides, pesticides, any other physiological process altering moieties, environmental pollutants, oxidation products, microbial metabolism products, viruses, prions, plasmids, bacteria, cells, yeast cells, fungi, cellular fragments, proteins, lipids, triglycerides, enzymes, proteins, minerals, heavy metals, organophosphates, biological warfare agents, chemical warfare agents, petrochemicals, soluble blood components, vitamins, antibodies, cytokines, growth factors, thyroid hormones, amine-containing metabolic breakdown products, amino acids, ketenes, liver toxins, ingested or inhaled toxins and poisons, tissue necrosis factors, heat shock proteins, anaphylactic shock factors, pharmaceuticals, drugs, biopharmaceuticals, drug analogs and derivatives and their metabolic products, antibiotics, anti-proliferative agents, anti-neoplastic agents, cancer drugs, anti-infective drugs, anti-inflammatory drugs, scar-inducing agents, neurotropic drugs, psychotropic medications, anesthetic and analgesic agents, anti-coagulative agents, pro-coagulative agents, radical quenchers, arsenates, cyanides, oncogenic compounds, carcinogenic compounds, cytokines, peptides, saccharides, polysaccharides, amino acids, nucleotides, pigments, immunomodulators, neuro-modulators, bone resorption products, implanted biomaterial breakdown products, implanted polymer and its degradation fragments, bacteria, infected blood cells, malignant or cancer cells, organic solvents, aromatic compounds, industrial pollutants, pollutant radicals, peroxides, and combinations, analogs, salts, ions, and/or derivatives thereof.

For example, the chemical target may be glucose, lactose, lactic acid, EDTA, DDT, paraquat, styrene, ethylene glycol, aldrin, calcium, magnesium, potassium, sodium, fluorine, cobalt, zinc, manganese, strontium, DNA, RNA, dopamine, serotonin, pyridinoline, deoxypyridinoline, pyridine, carbon tetrachloride, dimethyl nitrosamine, trichlorethylene, carbon monoxide, vitamin A, vitamin D, 17-hydroxyprogesterone, acetoacetate, amylase, ascorbic acid, bicarbonate, bilirubin, ceruloplasmin, chlorine, copper, creatine kinase, creatinine, hemoglobin, iron, lactic dehydrogenase, lead, lipase, zinc, cholesterol, oxygen, carbon dioxide, phosphatase, phosphorus, prostate-specific antigen, albumin, globulin, prothrombin, pyruvic acid, thyroid-stimulating hormone, alanine, aspartate, urea, and uric acid. Elemental chemical targets (e.g., chlorine, calcium, magnesium, etc.) may also be in the form of ions or salts.

### Binding Component

The binding component is generally a composition comprised of binding moieties capable of reversible binding, complexation, or other reversible interaction with a chemical target. The binding constants and capacity of the binding component may, for example, be selected to bind the chemical target under physiological conditions of the living body or the natural conditions of an ecosystem. Binding or dissociation of the chemical target to or from the binding component regulates the concentration of the chemical target in the body or ecosystem; the concentration of the chemical target may be reduced, elevated, or simply held constant as a result of this interaction.

Binding moieties that bind with the chemical targets described herein are generally well known, characterized, and documented in the literature.

Binding moieties will typically bind the chemical target through a reversible interaction. Reversible interactions include covalent bonding, hydrogen bonding, ionic bonding, Van der Waals interactions, and other forms of non-covalent bonding interactions.

The binding component may be comprised of a polymer or a combination of polymers. One or more binding moieties may be located along the backbone of the polymer, at the termini of the polymer, on sidechains of the polymer, or any combination thereof.

Hydrophilic polymers useful for making the binding component include: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines.

The hydrophilic polymers useful for preparing the binding component may be synthetic or naturally occurring hydrophilic polymers. Naturally occurring hydrophilic polymers include: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives.

Hydrophobic polymers, including low molecular weight polyfunctional species, can also be used in the binding component. Generally, hydrophobic polymers herein contain a relatively small proportion of heteroatoms such as O, S, P, N, or combinations thereof. Preferred hydrophobic polymers generally have carbon chains that are no longer than about 14 carbons between heteroatoms. Hydrophobic polymers are particularly useful for the binding component when slower biodegradation is desired. Polylactic acid and polyglycolic acid are examples of two hydrophobic polymers that can be used. The binding component may include a mixture of hydrophobic and hydrophilic polymers.

The binding component may also contain thermoreversible polymers, which may be useful in forming an implant in-situ. Hydrophobic interactions are often able to induce physical entanglement, especially in polymers, that induces increases in viscosity, precipitation, or gelation of polymeric solutions. For example, poly(oxyethylene)-poly(oxypropylene) block copolymers, available under the trade name of PLURONIC™ (BASF Corporation, Mount Olive, N.J.), are well known to exhibit a thermoreversible behavior in solution. Thus, an aqueous solution of 30% PLURONIC™ F-127 is a relatively low viscosity liquid at 4° C. and forms a pasty gel at physiological temperatures due to hydrophobic interactions. Other block and graft copolymers of water soluble and insoluble polymers exhibit similar effects, for example, copolymers of poly(oxyethylene) with poly(styrene), poly(caprolactone), poly(butadiene) etc. Recently, Jeong et al. reported biodegradable, in situ gelling poly(ethylene glycol-b-(DL-lactic acid-co-glycolic acid)-b-ethylene glycol), (PEG-PLGA-PEG), triblock copolymers. (See U.S. Pat. No. 6,117,949). They exhibited promising properties as an injectable drug delivery system. In vivo studies in rats demonstrated that the copolymer gels were still present after one month.

The binding component may comprise pH responsive polymers. These are polymers that have a low viscosity at acidic or basic pH, and exhibit an increase in viscosity upon reaching neutral pH, for example, due to decreased solubility. Thus, the stability and reactivity of crosslinkable aqueous polymers can be controlled by appropriate selection of pH. For example, degradable esters are stable at pH 3-5, while reactivity for electrophilic-nucleophilic reactions is highest at elevated pHs. For example, polyanionic polymers such as poly(acrylic acid) or poly(methacrylic acid) possess a low viscosity at acidic pHs that increases as the polymers become more solvated at higher pHs. The solubility and gelation of such polymers further may be controlled by interaction with other water soluble polymers that complex with the polyanionic polymers. For example, it is well known that poly(ethylene oxides) of molecular weight over 2,000 dissolve to form clear solutions in water. When these solutions are mixed with similar clear solutions of poly(methacrylic acid) or poly(acrylic acid), however, thickening, gelation, or precipitation occurs depending on the particular pH and conditions used. Thus, a two or more component aqueous solution system may be selected so that the first component comprises poly(acrylic acid) or poly(methacrylic acid) at an elevated pH of around 8-9 and another component comprises a solution of poly(ethylene glycol) at an acidic pH, such that the two solutions on being combined in situ result in an immediate increase in viscosity due to physical crosslinking.

The binding component may also comprise naturally occurring “environment-sensitive polymers”. Physical gelation may be obtained in several naturally existing polymers. For example, gelatin, which is a hydrolyzed form of collagen, gels by forming physical crosslinks when cooled from an elevated temperature. Other natural polymers, such as glycosaminoglycans, e.g., hyaluronic acid, contain both anionic and cationic functional groups along each polymeric chain, or covalently crosslinked hyaluronic acid. See, for example, X. Shu, Y. Liu, Y. Luo, M. Roberts, G. Prestwich, Biomacromolecules, 3: 1304-1311 (2002). This allows the formation of both intramolecular as well as intermolecular ionic crosslinks, and is responsible for the thixotropic (or shear thinning) nature of hyaluronic acid. The crosslinks are temporarily disrupted during shear, leading to low apparent viscosities and flow. The crosslinks reform on the removal of shear, thereby causing the gel to reform.

The synthetic hydrophilic polymers useful in the binding component may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade via the breakage of covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is one example. Poly(lysine)s have been prepared having between about 6 and about 4,000 primary amino groups, corresponding to molecular weights of about 870 to about 580,000. Poly(lysine)s for use in the compositions disclosed herein may have a molecular weight within the range of about 1,000 to about 300,000, for example within the range of about 5,000 to about 100,000, and as a further example, within the range of about 8,000 to about 15,000. Poly(lysine)s of varying molecular weights are commercially available from Peninsula Laboratories, Inc. (Belmont, Calif.).

Other examples of synthetic hydrophilic polymers useful in the binding component are hydroxyethylmethacrylate (HEMA), polyethylene glycol (PEG) and polyglycerol (PG). With respect to PEG, various functionalized polyethylene glycols have been used effectively in fields such as protein modification, peptide chemistry, and the synthesis of polymeric drugs. Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Ala. (1997-1998).

The binding component may also comprise thermogelling polymers. Examples of such polymers, and corresponding gelation temperatures (° C.), include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; and poly(N-ethylacrylamide), 72.0. In addition, the monomers that comprise these homopolymers may also be used to prepare thermogelling copolymers. Also within the scope of the present disclosure are combinations of thermogelling polymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X where X is a polyalkylene oxide and Y is a biodegradable polyester (e.g., PLG-PEG-PLG) and PLURONIC(S)™ such as F-127, 10-15° C.; L-122, 19° C.; L-92, 26° C.; L-81, 20° C.; and L-61, 24° C.

As discussed above, synthetic hydrophilic polymers that are useful for the making of binding compositions, particles and implants herein include, but are not limited to: polyalkylene oxides, particularly polyethylene glycol and poly(ethylene oxide)-poly(propylene oxide) copolymers, including block and random copolymers; polyols such as glycerol; polyglycerol (particularly highly branched polyglycerol); propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol; polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The binding component may include bioresorbable and/or biodegradable components. The compositions disclosed herein may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Representative examples of biodegradable polymers suitable for preparing the binding component include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, degradable polyesters, poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, d-decanolactone, trimethylene carbonate, 1,4-dioxane-2-one or 1,5-dioxepan-2-one), R is a multifunctional initiator and copolymers as well as blends thereof) and the copolymers as well as blends thereof.

Non-resorbable binding compositions are also within the scope of the invention, and are typically comprised of polymers that are substantially insoluble in physiologic liquids. Suitable biocompatible polymers include, by way of example, cellulose acetates (including cellulose diacetate), ethylene vinyl alcohol copolymers, hydrogels (e.g., acrylics), poly(C₁-C₆) acrylates, acrylate copolymers, polyalkyl alkacrylates wherein the alkyl groups independently contain one to six carbon atoms, polyacrylonitrile, polyvinylacetate, cellulose acetate butyrate, nitrocellulose, copolymers of urethane/carbonate, copolymers of styrene/maleic acid, and mixtures thereof. Copolymers of urethane/carbonate include polycarbonates that are diol terminated which are then reacted with a diisocyanate such as methylene bisphenyl diisocyanate to provide for the urethane/carbonate copolymers.

Representative examples of non-degradable polymers suitable for the binding component include poly(ethylene-co-vinyl acetate) (“EVA”) copolymers, non-degradable polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcyanoacrylate) (e.g., poly(ethylcyanoacrylate), poly(butylcyanoacrylate), poly(hexylcyanoacrylate), and poly(octylcyanoacrylate)), acrylic resin, polyethylene, polypropylene, polyamides (e.g., nylon 6 and nylon 6,6), polyurethanes (e.g., Chronoflex AR, Chronoflex AL, Bionate, and Pellethane), poly(ester urethanes), poly(ether urethanes), poly(ester-urea), cellulose esters (e.g., nitrocellulose), polyethers (e.g., poly(ethylene oxide) and poly(propylene oxide)), polyoxyalkylene ether block copolymers based on ethylene oxide and propylene oxide such as the PLURONIC™ polymers (e.g., F-127 or F87), and poly(tetramethylene glycol), styrene-based polymers (e.g., polystyrene, poly(styrene sulfonic acid), poly(styrene)-block-poly(isobutylene)-block-poly(styrene), and poly(styrene)-poly(isoprene) block copolymers), and vinyl polymers (e.g., polyvinylpyrrolidone, poly(vinyl alcohol), and poly(vinyl acetate phthalate)) as well as copolymers and blends thereof. Anionic polymers such as alginate, carrageenan, carboxymethyl cellulose, poly(acrylamido-2-methyl propane sulfonic acid), poly(methacrylic acid) and poly(acrylic acid), and cationic polymers such as chitosan, poly-L-lysine, polyethylenimine, and poly(allyl amine), as well as blends and copolymers thereof, are also suitable for use in the binding component.

Representative examples of patents describing other polymers suitable for preparing or delivering binding components include PCT Publication Nos. WO 98/19713, WO 01/17575, WO 01/41821, WO 01/41822, and WO 01/15526 (as well as the corresponding U.S. applications); U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,461,631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, 5,714,159, 5,612,052; and U.S. Patent Application Publication Nos. 2003/0068377, 2002/0192286, 2002/0076441, and 2002/0090398.

Furthermore, the binding component may comprise polymeric carriers which are temperature sensitive. See, for example, Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167, and Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27,1987, pp. 297-305.

The binding component may comprise, for example, a plurality of polymers that form a hydrogel upon mixing.

Copolymers, blends, mixtures, and composites of any of the aforementioned polymers and their constitutional monomer units are also suitable for the binding components of the compositions disclosed herein.

The binding component may comprise a variety of materials including organic salts, inorganic salts, ceramics, hydroxyapatite, tricalciumphosphate, solgels, organosilanes, sea coral, demineralized bone, glass, metal oxides (i.e. TiO₂), metals, lipids, polysaccharides, gold, silver, titanium, talc, Teflon, e-PTFE, Dacron, carbon, hydrogels, elastomers, plastics, metal alloys, cellulose, oxidized cellulose, polymers of drugs, silicone, antibodies or their analogs, or combinations and derivatives of thereof.

Within another aspect of the invention, the binding component can be prepared with a non-polymeric agent. These non-polymeric carriers can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof, sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof, sphingosine and derivatives thereof, spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites; and combinations and mixtures thereof. Such non-polymeric additives may be part of the binding component per se, or they may be a part of the binding composition in general (e.g., as transport agents, delivery vehicles, etc.).

Representative examples of patents relating to non-polymeric delivery systems and their preparation include U.S. Pat. Nos. 5,736,152; 5,888,533; 6,120,789; 5,968,542; and 5,747,058.

The binding components for use in the compositions disclosed herein will be selected based on a number of factors, including the identity and nature of the chemical target. Binding moieties for the chemical targets described herein are well-known in the art. For example, for chemical targets such as calcium or magnesium ions (e.g., Ca⁺² or Mg⁺²), the binding component may contain binding moieties such as carboxylate ions and the like.

The binding component for a composition according to the disclosure may have a single type of binding moiety or a plurality of binding moieties. Each binding moiety is capable of binding one or more chemical targets. These binding associations are equilibrium reactions. The binding moiety will uptake the target when the concentration of the target in the physiological fluid or ecosystem exceeds an equilibrium concentration. Conversely, the binding moiety will release the target when the concentration of the target in the physiological fluid or ecosystem falls below the equilibrium concentration. The equilibrium concentration is determined by the binding constant (discussed hereinbelow), which is an equilibrium constant that measures the strength with which a binding moiety binds to a particular chemical target.

The binding component may be prepared by standard organic synthetic techniques. The binding component may also be prepared by, for example, molecular imprinting, template polymerization, genetic engineering, or may be selected from synthetic combinatorial libraries.

### Other Components

The compositions disclosed herein may also contain various additives such as pharmaceutically acceptable carriers, transport agents, excipients, solubilizing agents, colorants, and visualization aids.

Pharmaceutically acceptable carriers are materials such as binders, lubricants, disintegrants, fillers, stabilizers, surfactants, coloring agents, and the like. Binders are used to impart cohesive qualities, and thus ensure that the composition remains intact (e.g., as an implant). Suitable binder materials include, but are not limited to, polymer matrices, hydrogels, starch (including corn starch and pregelatinized starch), gelatin, sugars (including sucrose, glucose, dextrose, and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate manufacture, promoting powder flow and preventing particle capping (i.e., particle breakage) when pressure is relieved. Useful lubricants are magnesium stearate, calcium stearate, and stearic acid. Disintegrants are used to facilitate disintegration of the composition, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard decomposition reactions that include, by way of example, oxidative reactions. The components of a composition may be distributed homogeneously throughout the pharmaceutically acceptable carrier, or localized regions of concentrations gradients may exist. Furthermore, components of a composition, such as the binding moieties, may be covalently or otherwise attached to the pharmaceutically acceptable carrier.

The compositions disclosed herein may contain a contrast agent such as a biocompatible (non-toxic) radiopaque material. Such a material may be capable, for example, of being monitored by radiography during injection into a mammalian subject. The contrast agent can be either water soluble or water insoluble. Examples of water soluble contrast agents include metrizamide, iopamidol, iothalamate sodium, iodomide sodium, and meglumine. Water insoluble contrast agents may have a water solubility of less than 0.01 milligrams per milliliter at 20° C., and include tantalum, tantalum oxide and barium sulfate, each of which is commercially available in a form suitable for in vivo use and preferably having a particle size of 10 micrometers or less. Other water insoluble contrast agents include gold, tungsten and platinum powders.

In one embodiment of a composition suitable for use in treating the physiological fluid of an organism, the compositions described herein comprise pharmaceutical agents, growth factors, proteins, and other physiological fluid components. These components may be released into the physiological fluid due to resorption of the composition. Such compositions may exhibit beneficial tissue healing and tissue regeneration effects.

Within certain embodiments of the invention, the composition can also comprise radio-opaque, echogenic materials and magnetic resonance imaging (MRI) responsive materials (i.e., MRI contrast agents) to aid in visualization of the composition under ultrasound, fluoroscopy and/or MRI. For example, the composition may be made in the form of a device containing or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexyl, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a device by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium (III) chelates or iron oxide compounds) may be incorporated into or onto the device, such as, for example, as a component in a coating or within the void volume of the device (e.g., within a lumen, reservoir, or within the structural material used to form the device). In some embodiments, a medical device may include radio-opaque or MRI visible markers (e.g., bands) that may be used to orient and guide the device during the implantation procedure.

The compositions described herein may, alternatively or in addition, be visualized under visible light, using fluorescence, or by other spectroscopic means. Visualization agents that can be included for this purpose include dyes, pigments, and other colored agents. In one aspect, the composition may further include a colorant to improve visualization in an ecosystem, in vivo, and/or ex vivo. Frequently, for example, implants can be difficult to visualize upon insertion, especially at the margins of the implant. A coloring agent can be incorporated into an implant comprising a composition of the invention to reduce or eliminate the incidence or severity of this problem. The coloring agent provides a unique color, increased contrast, or unique fluorescence characteristics to the device. In one aspect, a solid implant is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan. Derivatives, analogs, and isomers of any of the above colored compound may also be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the composition of the present invention include one or more coloring agents, also referred to as dyestuffs, which will be present in an effective amount to impart observable coloration to the composition, e.g., the gel. Examples of coloring agents include dyes suitable for food such as those known as F.D. & C. dyes and natural coloring agents such as grape skin extract, beet red powder, beta carotene, annato, carmine, turmeric, paprika, and so forth. Derivatives, analogs, and isomers of any of the above colored compound may also be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one embodiment, the binding compositions disclosed herein comprises magnetic components such as magnetite, stabilized magnetic ferro-colloids, or magnetic fluids. Such magnetic field-responsive materials can aid in concentration and removal of the compositions disclosed herein from the organism or ecosystem.

In yet another embodiment, gold particles and colloids can be used as a part of the compositions disclosed herein. For example, commercially available gold particles ranging in size between 1 nm and 1000 nm can be coated with the binding component. Gold particles provide high total surface area for quick binding or release of the chemical target. In one specific embodiment, an implant may be prepared using a gold particle suspension with a glucose-binding component, and the composition can be placed in an immunoisolation-like device used to cultivate transplanted islet cells in vivo. The immunoisolation device protects the gold particles and glucose binding surfaces from direct contact with cells, particularly macrophages, thus increasing the functional longevity of the implant.

In one aspect, the compositions of the present invention include one or more preservatives or bacteriostatic agents, present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, 5-fluorouracil, methotrexate, doxorubicin, mitoxantrone, rifamycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the compositions of the present invention include one or more antioxidants, present in an effective amount. Examples of the antioxidant include sulfites, alpha-tocopherol and ascorbic acid.

The compositions disclosed herein or the precursors thereof may further contain porosifying agents that achieve greater surface area of, for example, an implanted metabolite-binding composition and faster diffusion and chemical target equilibrium between the metabolite-binding composition and the living body or ecosystem. Examples of porosifying agents include inorganic salts, sucrose, surfactants, small molecular weight polymers, fast degrading polymers, thermoreversible polymer precipitates, gas bubbles, and cavitation bubbles.

Within certain embodiments of the invention, the compositions may also comprise additional ingredients such as surfactants (e.g., PLURONICS™, such as F-127, L-122, L-101, L-92, L-81, and L-61), anti-inflammatory agents (e.g., dexamethasone or aspirin), anti-thrombotic agents (e.g., heparin, high activity heparin, heparin, and quaternary amine complexes such as heparin benzalkonium chloride complex), anti-infective agents (e.g., 5-fluorouracil, triclosan, rifamycim, and silver compounds), preservatives, anti-oxidants and/or anti-platelet agents.

Other carriers that may likewise be utilized to contain and deliver the compositions disclosed herein include: hydroxypropyl cyclodextrin, liposomes, liposome/gel, nanocapsules, micelles, implants, nanoparticles, nanoparticles having modified surface, micelle (surfactant), synthetic phospholipid compounds, gas borne dispersion, liquid emulsions, foam, spray, gel, lotion, cream, ointment, dispersed vesicles, particles or droplets solid- or liquid-aerosols, microemulsions, polymeric shell (nano- and micro-capsule), emulsion, nanospheres and implants.

Any suitable solvent may be used to prepare and/or deliver the compositions described herein to an organism or ecosystem; such solvents are well known in the art, and the choice of solvent will be apparent to those skilled in the art. Suitable biocompatible solvents that can be used to deliver non-water soluble binding compositions into the body or ecosystem include, by way of example, dimethylsulfoxide, analogs/homologues of dimethylsulfoxide, ethanol, ethyl lactate, acetone, and the like. Aqueous mixtures with the biocompatible solvent can also be employed provided that the amount of water employed is sufficiently small that the dissolved metabolite-binding composition precipitates upon injection into a human body. Preferably, the biocompatible solvent is ethyl lactate or dimethylsulfoxide.

Yet in another aspect of this invention the binding composition comprises a drug or other entity used to alter a biological process locally or systemically. Such drug or entity may be covalently attached to the binding component, entrapped within the binding component by polymer or retained with the binding component by charge, hydrophobic or other molecular interactions. In more specific embodiments such drug or entity can have anti-infective, anti-inflammatory, anti-proliferative, anti-scarring, anti-adhesive, anti-neoplastic, immuno-modulating, analgesic, scar-forming, tissue-regenerative and tissue-repair promoting effects.

### Structures, Devices, Dosage Forms

The compositions described herein may be incorporated into any appropriate dosage form for delivery to an organism or to an ecosystem, such dosage forms being well known in the art. In addition to dosage forms that have no defined structure, such as a solution, the compositions may be formed into a variety of different structures or devices.

As described above, a range of polymeric and non-polymeric materials can be used to incorporate the binding composition onto or into a dosage form or device. The binding composition can be incorporated into or onto the device in a variety of ways. The binding composition may be coated onto the entire device or a portion of the device using a method, such as by dipping, spraying, painting or vacuum deposition that is appropriate for the particular type of device. The device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

For compositions intended to be used in vivo, the compositions disclosed herein may include a fibrosis-inhibiting agent and an anti-thrombotic agent and/or antiplatelet agent and/or a thrombolytic agent, which reduces the likelihood of thrombotic events upon implantation of a medical implant. Within various embodiments of the invention, a device is coated on one aspect with a composition which inhibits fibrosis (and/or restenosis), as well as being coated with a composition or compound which prevents thrombosis on another aspect of the device. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

The thrombogenicity of a binding composition may be reduced by coating the implant with a polymeric formulation that has anti-thrombogenic properties. For example, a medical device may be coated with a metabolite-binding hydrophilic polymer gel. The polymer gel can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC™ polymers (e.g., F-127 or F87), chain extended PLURONIC™ polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Other examples of implantable biomaterials that can be used to prepare binding compositions in this invention include sprayable collagen-containing formulations such as VitaGel (from Orthovita, Inc. Marven, Pa.), sprayable PEG-containing formulations such as CoSeal (Baxter Healthcare Corporation, Deerfield, Ill.), SprayGel or DuraSeal (both from Confluent Surgical, Inc., Boston, Mass.), FocalSeal (Genzyme Corporation, Cambridge, Mass.), fibrin-containing formulations such as FloSeal or Tisseel (both from Baxter Healthcare Corporation, Deerfield, Ill.), hyaluronic acid-containing formulations such as Restylane or Perlane (both from Q-Med AB, Sweden), Hylaform (Inamed Corporation (Santa Barbara, Calif.)), Synvisc (Biomatrix, Inc., Ridgefield, N.J.), Seprafilm or Sepracoat (both from Genzyme Corporation, Cambridge, Mass.), Intergel (Lifecore Biomedical), polymeric gels for surgical implantation such as Repel (Life Medical Sciences, Inc., Princeton, N.J.) or Flogel (Baxter Healthcare Corporation), orthopedic “cements” used to hold prostheses and tissues in place with a fibrosis-inhibiting agent applied to the implantation site (or the implant/device surface); surgical adhesives containing cyanoacrylates such as Dermabond (Johnson & Johnson, Inc., New Brunswick, N.J.), Indermil (U.S. Surgical Company, Norwalk, Conn.), Glustitch (Blacklock Medical Products Inc., Canada), Tissumend II (Veterinary Products Laboratories, Phoenix, Ariz.), Vetbond (3M Company, St. Paul, Minn.), Histoacryl Blue (Davis & Geck, St. Louis, Mo.) and Orabase Smoothe-N-Seal Liquid Protectant (Colgate-Palmolive Company, New York, N.Y.), surgical implants containing hydroxyapatite, calcium sulfate, tricalcium phosphate, demineralized bone loaded with a metabolite-binding composition applied to the implantation site (or the implant/device surface).

The compositions described herein may incorporate an angiogenic layer to promote vascularization. For an implantable binding composition to be effective long-term (months or years), an implant/tissue interface must be created which provides quick diffusion equilibrium between the chemical target in the peripheral blood and the binding composition. The achievement of rapid equilibrium for the binding composition in the living body or in the ecosystem is particularly important for vital chemical targets such as glucose or certain drugs and toxic chemical targets such as toxins. The proximity of blood capillaries to the surface of the metabolite-binding composition is essential for achieving rapid equilibrium between the binding composition and the blood circulation. The development of such blood/implant interfaces in other contexts has been reported. For example, investigators have developed techniques that stimulate and maintain blood vessels inside a foreign body capsule formed around the implant to provide for the demanding oxygen needs of pancreatic islets within an implanted membrane (See, e.g., Brauker et al., J. Biomed. Mater. Res. (1995) 29:1517-1524) and Published US Patent Application No. 2005/0177036).

In one embodiment for the purposes of this invention, a binding composition can comprise or be coated with a material causing microvascular ingrowth in vivo. Among materials known to cause the formation of stable microvasculature in the structure of the material is e-PTFE material or porous polyvinylpyrrolidone.

In another embodiment, the outermost layer of a binding composition such as an implant includes an angiogenic material. The angiogenic layer of the compositions of the present invention may be constructed of membrane materials such as hydrophilic polyvinylidene fluoride (e.g., Durapore®; Millipore Bedford, Mass.), mixed cellulose esters (e.g., MF; Millipore Bedford, Mass.), polyvinyl chloride (e.g., PVC; Millipore Bedford, Mass.), and other polymers including, but not limited to, polypropylene, polysulfone, and polymethylmethacrylate. Preferably, the thickness of the angiogenic layer is about 10 micrometers to about 50 micrometers. The angiogenic layer comprises pores sizes of about 0.5 micrometers to about 20 micrometers, for example about 1.0 micrometers to about 10 micrometers, sizes that allow most substances to pass through, including, e.g., macrophages. One specific example material is expanded PTFE of a thickness of about 15 micrometers and pore sizes of about 5 micrometers to about 10 micrometers.

To further promote, for example, a stable foreign body capsule structure without interfering with angiogenesis, an additional outermost layer of material comprised of a thin low-density non-woven polyester (e.g., manufactured by Reemay, Inc.) can be laminated over the angiogenic layer (e.g., PTFE) described above. In one embodiment, the thickness of this layer is about 120 micrometers. This additional thin layer of material does not interfere with angiogenesis and enhances the manufacturability of the angiogenic layer. Examples of such angiogenic and protective layer materials are described in U.S. Pat. Nos. 5,741,330, 5,782,912, 5,800,529, 5,882,354 5,964,804 and Published US Patent Application No. 2005/0177036, and hereby are incorporated by reference.

The compositions described herein may contain a bioprotective material such as a bioprotective membrane. Example bioprotective materials include polyurethane, polytetrafluoroethylene, polypropylene, polyethylene, and polysulfone. The inflammatory response that initiates and sustains a foreign body response is associated with both advantages and disadvantages. Some inflammatory response may be beneficial to create a new capillary bed in close proximity to the surface of the binding composition. On the other hand, inflammation is associated with invasion of tissue macrophages that have the ability to biodegrade many artificial biomaterials (some of which were, until recently, considered non-biodegradable). When activated by a foreign body, tissue macrophages degranulate, releasing from their cytoplasmic myeloperoxidase system hypochlorite (bleach), H₂O₂ and other oxidant species. Both hypochlorite and H₂O₂ are known to break down a variety of polymers, including polyurethane, by a phenomenon referred to as environmental stress cracking.

Because both hypochlorite and H₂O₂ are short-lived chemical species in vivo, biodegradation will not occur if macrophages are kept a sufficient distance from the enzyme active membrane. In one embodiment, the present invention contemplates the use of a bioprotective membrane that allows transport of a chemical target but prevents the entry of inflammatory cells such as macrophages and foreign body giant cells. The bioprotective membrane is placed proximal to the angiogenic membrane, when one is present. It may be simply placed adjacent to the angiogenic layer without adhering, or it may be attached with an adhesive material to the angiogenic layer, or it may be cast in place upon the angiogenic layer. The devices of the present invention are not limited by the nature of the bioprotective layer.

The bioprotective membrane and the angiogenic layer, when both are present, may be combined into a single bilayer membrane as described by way of example in published US Patent Application No. 2005/0124873 and incorporated herein by reference. The active angiogenic function of the combined membrane is based on the presentation of the e-PTFE side of the membrane to the reactive cells of the foreign body capsule and further to the response of the tissue to the microstructure of the e-PTFE. Although the physical structure of the e-PTFE represents one embodiment, many other combinations of materials that provide the same function. For example, the e-PTFE could be replaced by other fine fibrous materials. In particular, polymers such as spun polyolefin or non-organic materials such as mineral or glass fibers may be useful.

The compositions described herein may further be contained within a container. Such a container may have, for example a semi-permeable or permeable boundary layer that prevents the composition from losing cohesiveness, but does not prevent transport of the chemical target (or other components of the environment or composition) into or out of the composition. Therefore, the pore sizes in the boundary layer will be selected to ensure such transport. Semi-permeable and permeable membranes with varying pore sizes are well known in the art. Alternatively, the boundary layer may be impermeable. In such a case, the container will preferentially be configured such that the boundary layer is breached and the binding composition is released (either wholly or in portions) from the container once the container is placed in the ecosystem or in contact with the physiological fluid.

Activated PEG implants and particles may be prepared in various ways. In one of the approaches a commercially available PEG polymer having a pentaerythritol (2,2-bis(hydroxymethyl)-1,3-propanediol) core and molecular weight of approximately 10,000 Da (prepolymer) can be crosslinked into a three-dimensional polymer by reaction with glutaryl dichloride in the presence of pyridine as a base. The crosslinked PEG can be formed into an implant of a desired shape or into particles of different sizes by mechanical grinding after the crosslinking step or by conducting the crosslinking process in a micellar system where a prepolymer is crosslinked in micelles of the desired size. The crosslinking process conditions can be optimized such that the formed particles contain carboxyl groups in the form of glutaryl groups. Alternatively, additional carboxyl groups can be readily prepared by conversion of the exposed, unreacted hydroxyl groups to carboxylic acid groups using a reaction with an anhydride in the presence of a nitrogenous base. The carboxyl groups on the particles can be activated by esterification with N-hydroxysuccinimide, N-hydroxysulfosuccinimide, or the like, to prepare polyfunctionally activated PEG implants or particles. Yet other forms of activated particles are functionally activated PEG glycidyl ether particles, PEG-isocyanate particles, and PEG-vinylsulfone particles.

Further examples of polymers, crosslinking reagents, activation chemistries and reaction conditions suitable for forming a binding composition for the purposes of this invention include those described in U.S. Pat. Nos. 6,312,725; 6,352,710; 6,217,894; 6,818,018; 6,833,408, and U.S. Patent Applications 20040219214, 20040225077, 20040009205, 20030162841, 20030104032, 20040002456, 20020114775.

In one embodiment, the binding compositions described herein may be prepared in the form of an implant.

Binding compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment.

Within yet other aspects of the invention, the binding compositions of the present invention may be formed as a film or tube. These films or tubes can be porous or non-porous. Preferably, such films or tubes are generally less than 5, 4, 3, 2, or 1 mm thick, more preferably less than 0.75 mm, 0.5 mm, 0.25 mm, or, 0.10 mm thick. Films or tubes can also be generated of thicknesses less than 50 micrometers, 25 micrometers or 10 micrometers. Such films are preferably flexible with a good tensile strength (e.g., greater than 50, preferably greater than 100, and more preferably greater than 150 or 200 N/cm²), good adhesive properties (i.e., adheres to moist or wet surfaces), and have controlled permeability.

The binding compositions described herein can be formulated to be biodegradable with a half-degradation time ranging from 1 day to 12 months. The rate of biodegradation can be modulated by incorporating into the composition hydrolysable or biodegradable chemical bonds and biodegradable segments mention herein and well known in the art. Typically, hydrogel materials containing up to 30% of water by weight are biodegraded more rapidly than elastomeric materials containing less then 50% of water by weight. Longer lasting elastomeric materials such as cellular perfluoroelastomers from CuMedica, Ltd. UK or a non-degradable material, Enteryx™, from Boston Scientific are examples of longer lasting biomaterial implants potentially suitable for the binding compositions.

### Methods of Use

The binding composition can be associated with a medical device or other device or dosage form suitable for administration to an organism or ecosystem using the polymeric carriers or coatings described herein. In addition to the compositions and methods described above, there are various other compositions and methods that are known in the art. Representative examples of these compositions and methods for applying (e.g., coating) these compositions to devices are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. patent application Publication Nos. 2002/0146581, 2003/0129130, 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

Generally, the binding compositions disclosed herein are suitable for use as physiological and/or environmental buffer compositions. As a physiological buffer, the binding composition is useful in regulating the concentration of a chemical target in a physiological fluid. Pharmaceutical compositions as disclosed herein are generally administered in a pharmaceutically acceptable dosage form. As an environmental buffer, the binding composition may be introduced into an ecosystem in order to regulate the concentration of a chemical target therein. Environmental formulations comprising the binding composition are generally introduced in a form that is environmentally compatible and appropriate for achieving this goal. In many instances, such as tablets, hydrogels, solutions, particles, and beads, an environmental formulation may be very similar to a pharmaceutical dosage form. Differences, such as tablet size, carrier matrix, solvent composition, and appropriate additives, will be apparent to one of skill in the art.

Regulation of the concentration of a chemical target, whether in a physiological fluid or in an ecosystem, may involve increasing or decreasing the concentration from an relatively low or relatively high initial value. Regulation may also involve maintaining the concentration substantially within some predetermined and desirable range of concentrations.

Depending on the intended mode of administration, a pharmaceutical composition may be a solid, semi-solid, or liquid, such as, for example, a tablet, a capsule, caplets, a liquid solution, a suspension, an emulsion, a gel, a suppository, granules, particles, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature. See, e.g., Remington: The Science and Practice of Pharmacy, 19th Ed. (Easton, Pa.: Mack Publishing Co., 1995). The compositions may be in the form of a hydrogel, hydrogel particles, hydrogel beads, or a hydrogel paste. The compositions may be in the form of a bolus, either for implantation or injection.

Pharmaceutical compositions disclosed herein may be suitable for administration to a patient parenterally, orally, rectally, vaginally, sublingually, nasally, topically, or transdermally. The composition may also be suitable to be implanted into a patient. Such implantation procedures may involve implantation into, for example, muscle tissue, subcutaneous tissue, bone, liver tissue, the peritoneal cavity, the thoracic cavity, a blood vessel, lung tissue, brain tissue, the cerebrospinal canal, eye tissue, kidney tissue, spleen tissue, fatty tissue, or bladder tissue

Compositions useful as environmental buffers may be introduced to the ecosystem in the form of a solid, semi-solid, or liquid, such as, for example, a tablet, a capsule, caplets, a liquid, a suspension, an emulsion, a gel, granules, particles, pellets, beads, a powder, or the like.

In one embodiment, polymers carrying a binding component can be formulated as aqueous solutions for delivery into the body or ecosystem. The binding component can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethylcellulose (CMC), and the like). In another aspect of the invention, the solution can include a biocompatible solvent, such as ethanol, DMSO, glycerol, PEG-200, PEG-300 or NMP.

The binding compositions described herein may also be formulated as particles. Particles can be prepared by covalently crosslinking monomers or prepolymers, by condensation from reactive prepolymers, by charge interaction, by hydrophobic interaction, by physical crosslinking, by temperature precipitation, by solvent precipitation, by co-precipitation with other component, by drying, by freeze-drying, by phase separation, by emulsification, by sonication, by extrusion, by spray-drying, by denaturation and by many other methods known in the art.

Therapeutic compositions and devices described herein should preferably have a stable shelf-life for several months and capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance from Cobalt 60 source and electron beam. A typical dose of gamma radiation is 2.5 MRad. Filtration may be accomplished using a filter with suitable pore size, for example 0.22 micrometers and of a suitable material, for instance polytetrafluoroethylene (e.g., Teflon from E.I. DuPont De Nemours and Company, Wilmington, Del.).

In another aspect, the binding compositions and devices of the present invention are contained in a container that allows them to be used for their intended purpose, e.g., as a pharmaceutical composition or in a device for regulating environmental variables. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), and an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through II and Type NP glass (refer to USP XXII <661>), polyethylene, TEFLON, silicone, and gray-butyl rubber.

In one embodiment, the product containers can be thermoformed plastics. In another embodiment, a secondary package can be used for the product. In another embodiment, the product can be in a sterile container that is placed in a box that is labeled to describe the contents of the box.

Within one aspect of the present invention, the binding compositions are used in vivo for regulation of a component of a physiological fluid, and any component of the binding compositions can be formed in-situ. For example, a polymeric binding component may form in situ, and the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Materials that undergo polymerization (e.g., free radical polymerization) and would be suitable for such uses are well know in the art.

In another embodiment, the metabolite-binding composition may be formed from reagents that can undergo an electrophilic-nucleophilic reaction to produce a crosslinked matrix. For example, a 4-armed thiol derivatized polyethylene glycol can be reacted with a 4 armed NHS-derivatized polyethylene glycol under basic conditions (pH> about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; PCT Application Published Nos. WO 04/060405 and WO 04/060346. Other examples of in situ forming materials that can be used include those based on the crosslinking of proteins (described in U.S. Pat. Nos. RE38158; 4,839,345; 5,514,379, 5,583,114; 6,458,147; 6,371,975; U.S. Publication Nos. 2002/0161399; 2001/0018598 and PCT Publication Nos. WO 03/090683; WO 01/45761; WO 99/66964 and WO 96/03159).

In one embodiment of this invention, the binding composition delivered into the body or ecosystem is composed of two reactive polymers able to form a hydrogel or elastomer within 1 sec to 20 min. The two reactive polymers are mixed during delivery of the binding composition. For example, a Y-shaped connector and mixer may be used, wherein soluble reactive reagents are placed into syringes connected to the Y-connector having a mixing chamber. Upon extrusion of the reactive reagents from syringes, the reagents are mixed with each other as the mixture is delivered onto the target tissue site. An optional gas source (nitrogen, carbon dioxide, compressed air) can be connected to the Y connector to deliver the metabolite-binding composition in the form of an aerosol. The reaction conditions (such as pH, polymerization initiator concentration, reactive reagent concentration) can be set to allow immediate (less then 10 sec) polymerization or gelation or slow (up to 20 min or longer) polymerization or gelation of the binding composition.

Yet in a different embodiment, the reactive reagents can be premixed immediately prior to application of the admixture to the tissue site or ecosystem. For example, the reactive agents may be premixed and delivered in a suitable body cavity in the form of a single reagent. The reaction conditions can be set to allow the admixture to remain liquid and extrudable for several minutes, which, typically, is sufficient for completing the delivery of the admixed material into the body or ecosystem.

In another aspect of this invention, one or more reactive reagents are also reactive with nucleophilic groups on the tissue surface (e.g., amino and sulfhydril groups of proteins on the cell surface and extracellular matrix). Upon delivery, the admixed reactive reagents react with each other and form the binding biomaterial implant. Some of the electrophilic groups react with nucleophilic groups on the tissue surface forming covalent chemical links between the metabolite-binding implant and the tissue surface. For example, if such a reactive formulation is delivered into the peritoneal space and placed in contact with peritoneal tissue surface, the implanted metabolite-binding composition can covalently link to the peritoneal tissue surface. Several commercial products in the field of biosurgery, including implantable sealants and adhesives such as CoSeal®, DuraSeal™ and BioGlue™, are based on crosslinking chemistries that also allow the covalent linking of the implant with the tissue surface.

In another aspect of the invention binding compositions can be prepared to contain reactive groups, or groups that can be further modified or activated to render said groups reactive. Among such groups are carboxylic acid groups. Since a carboxylic acid group per se may not be susceptible to reaction with a nucleophilic amine or a sulfhydryl, components containing carboxylic acid groups must be activated so as to be amine-, or sulfhydryl-reactive. For example, a carboxylic acid can be reacted with an alkoxy-substituted N-hydroxy-succinimide or N-hydroxysulfosuccinimide in the presence of DCC to form the reactive electrophilic groups N-hydroxysuccinimide ester and N-hydroxysulfosuccinimide ester, respectively. Carboxylic acids may also be activated by reaction with an acyl halide such as acyl chloride (e.g., acetyl chloride), to provide a reactive anhydride group. In a further example, a carboxylic acid may be converted to an acid chloride group using a thionyl chloride or an acyl chloride capable of an exchange reaction. Specific reagents and procedures used to carry out such activation reactions will be known to those of ordinary skill in the art and are described in pertinent texts and literature. The activated electrophilic groups can react with the nucleophilic groups on the tissue surface or nucleophilic groups present on the tissue surface. When the nucleophilic groups on the tissue surface are sulfhydryls, the reactive electrophilic groups include those that form thioester linkages upon reaction with the sulfhydryl group. Such sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, for example 1-ethyl-3-[3-dimethylaminopropyl]carbodiimide can be used to facilitate the coupling of sulfhydryl groups to carboxyl-containing groups. In addition to the sulfhydryl reactive groups that form thioester linkages, various other sulfhydryl reactive functionalities can be utilized to form other types of linkages. For example, compounds that contain methyl imidate derivatives form imido-thioester linkages with sulfhydryl groups.

Other examples of sulfhydryl reactive groups are those forming thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and alpha-, beta-unsaturated aldehydes and ketones. This class of sulfhydryl reactive groups is particularly useful because the thioether bonds may provide faster crosslinking and longer in vivo stability.

In addition to the sulfhydryl reactive compounds that form thioester linkages, various other compounds can be utilized that form other types of linkages. For example, compounds that contain methyl imidate derivatives form imido-thioester linkages with sulfhydryl groups. Alternatively, sulfhydryl reactive groups can be employed to form disulfide bonds with sulfhydryl groups, such as ortho pyridyl disulfide, 3-nitro-2-pyridenesulfenyl, 2-nitro-5-thiocyanobenzoic acid, 5,5′-dithio-bis(2-nitrobenzoic acid), derivatives of methane-thiosulfate, and 2,4-dinitrophenyl cysteinyl disulfides. In such instances auxiliary reagents such as hydrogen peroxide or the di-tert-butyl ester of azodicarboxylic acid can be used to facilitate disulfide bond formation. Yet another class of sulfhydryl reactive groups form thioether bonds with sulfhydryl groups. Such groups include, inter alia, iodoacetamide, N-ethylmaleimide and other maleimides, including dextran maleimides, mono-bromo-bimane and related compounds, vinylsulfones, epoxides, derivatives of O-methyl-isourea, ethyleneimines, and aziridines.

Other general principles should be considered with respect to linking groups used in preparation of binding compositions. For example, if higher molecular weight components are to be used, it is preferred that they have biodegradable linkages as described above, so that fragments larger than 15,000 Daltons are not generated during resorption in the body. In addition, to promote water miscibility and/or solubility, it may be desired to add sufficient electric charge or hydrophilicity. Hydrophilic groups can be easily introduced using known chemical synthesis, as long as they do not give rise to unwanted swelling or an undesirable decrease in compressive strength. In particular, polyalkoxy segments may weaken gel strength.

For binding compositions that incorporate polymeric binding components and/or carrier components, the binding component (whether polymeric or not) may be linked to the polymer by covalent bonds formed between reactive moieties present on chemical target binding moieties and reactive groups on the polymer, or by occlusion in the matrices of the polymer, or encapsulated in polymeric microcapsules. Within certain embodiments of the invention, therapeutic compositions are provided in non-capsular formulations such as microspheres (ranging from nanometers to micrometers in size), pastes, threads of various size, films, or sprays. In one aspect, an anti-scarring agent may be incorporated into biodegradable magnetic nanospheres. The nanospheres may be used, for example, to replenish target-binding moieties into an implanted intravascular device, such as a stent containing a weak magnetic alloy (see, e.g., Z. Forbes, B. B. Yellen, G. Friedman, K. Barbee. “An approach to targeted drug delivery based on uniform magnetic fields,” IEEE Trans. Magn. 39(5): 3372-3377 (2003)). Within certain aspects of the present invention, therapeutic compositions may be fashioned in the form of microspheres, microparticles and/or nanoparticles having any size ranging from about 30 nm to 500 micrometers, depending upon the particular use. These compositions can be formed by spray-drying methods, milling methods, coacervation methods, W/O emulsion methods, W/O/W emulsion methods, and solvent evaporation methods. In other aspects, these compositions can include microemulsions, emulsions, liposomes and micelles. Alternatively, such compositions may also be readily applied as a “spray”, which solidifies into a film or coating for use as a device/implant surface coating or to line the tissues of the implantation site.

The binding composition described herein and for which the chemical target is a drug may also be used to prolong drug release of the drug. In this case, the drug typically is injected or introduced into the body by known techniques such as intra-venous, intra-peritoneal, intra-muscular, sub-cutaneous, orally, by inhalation or otherwise. Drug is bound by the binding composition while it is present in the body at a high concentration following the bolus injection or intake of the drug. The drug is released by dissociation from the bound form when free drug concentration in the body is lower than the peak concentration or after bolus injection or intake of the drug. When the binding composition is an implant, for example, the drug may be injected or introduced into the body at a location that is either relatively distant or relatively near to the location of the binding composition implant. This method may also be used, for example, to regulate the concentration of the chemical target in the body. In such a circumstance, the binding composition uptakes (i.e., binds) the chemical target when the target is present in the body at a concentration that is higher than the equilibrium concentration for the implant. Conversely, the binding composition releases the chemical target when the target is present in the body at a concentration that is lower than the equilibrium concentration for the implant. It will be appreciated, therefore, that the equilibrium concentration and reservoir capacity for the chemical target in the binding composition must be appropriately chosen for the particular application intended. Factors that affect the equilibrium concentration and reservoir capacity are well known and well characterized in the art, and include binding constants and the quantity of binding sites in the composition.

In one embodiment, drugs, biopharmaceuticals, or any other physiological process modifying agents are concentrated at the local tissue after the drug is introduced into the body. Such increase in local concentration of the drug or agent in the proximity of the drug-binding implant occurs due to the reversible binding of the drug with the drug-binding implant. The biomaterial delivered drug, biopharmaceuticals, therapeutic agents or physiological process modifying agents can be anti-infective, anti-inflammatory, anti-proliferative, anti-angiogenic, anti-neoplastic, anti-scaring, scar-inducing, tissue-regenerative, anesthetic, analgesic, immuno-modulating agents and neuro-modulating. Further examples of drugs include those described in U.S. Pat. No. 6,759,431, and U.S. Patent Applications 20040219214, 20050177225 which are thereby incorporated by the reference.

Whether a composition is used to regulate the concentration of a chemical target in a physiological fluid or in an ecosystem, important factors for consideration include the binding constant(s) of the binding moieties and the quantity of the binding moieties in the composition. The binding constant relates to the equilibrium concentration of the chemical target, and is therefore important in determining the concentration at which there will be no further net uptake or release of the chemical target into or out of the composition (and, conversely, no further net outflow or inflow of the chemical target from or into the physiological fluid or ecosystem). The quantity of binding moieties in a composition may be influenced by a number of factors, such as volume, mass, density, surface area, porosity, and the number of binding moieties per molecule of the binding component.

In one embodiment, the compositions disclosed herein are useful in maintaining the concentration of a chemical target in a physiological fluid or ecosystem substantially within a beneficial range of concentrations. For metabolites and other normal components of blood, for example, beneficial ranges of concentrations corresponding to physiologically acceptable levels are known in the art. For the delivery of drugs to a patient's blood or other physiological fluids, beneficial ranges of concentrations are determined by the recommended dosage for the individual drug. For the regulation of toxins in an ecosystem, beneficial ranges will typically be maximum concentrations wherein the health of the resident organisms are not adversely affected by the toxin. For the regulation of nutrients in an ecosystem, beneficial ranges will typically be minimum concentrations below which resident organisms are not able to find sufficient nourishment. Beneficial ranges of concentrations for each of these situations, as well as others, are known in the art.

The beneficial range of concentrations that is determined for any particular situation (e.g., concentrations of a metabolite in blood) is useful for determining the preferred binding constant of the binding moieties. Selection of appropriate binding moieties is therefore based on known or determined binding constants for a particular moiety with a particular chemical target. The binding constant of a suitable binding moiety will preferentially be complementary to the beneficial range of concentrations for the chemical target in the environment in which it is to be regulated. That is, a complementary binding constant is such that, in the binding composition, the equilibrium concentration of the chemical target is within the beneficial range of concentrations. The binding composition is thereby suitable for maintaining the concentration of the chemical target substantially within the beneficial range of concentrations.

As an example, the beneficial concentration of Ca⁺² in blood plasma of an average human is 2.5 mM. A complementary binding constant for the binding moieties of a suitable Ca⁺²-binding composition would be one in which the Ca⁺²-binding composition is at equilibrium with a physiological fluid containing Ca⁺² in a concentration of 2.5 mM. That is, the binding composition has no net uptake or release of Ca⁺² when the surrounding environment contains a Ca⁺² concentration of 2.5 mM.

Beneficial ranges of concentrations for blood analytes are well known in the art. For example, see Stedman's Medical Dictionary, 26^{th} edition (Williams & Wilkins, Baltimore, Md., 1995).

The required quantity of binding moieties in a composition according to the disclosure will be readily determined by one of ordinary skill. For example, an average adult human may have 5-6 liters of blood, with a glucose concentration of approximately 0.8 mg/mL. Regulation of the concentration of glucose in this quantity of blood at this concentration requires a sufficient dosage of a glucose-binding composition, determination of such dosage being within the grasp of one of skill in the art.

The compositions disclosed herein may be capable of regulating the concentration of a chemical target in a physiological fluid or ecosystem for a predetermined period of time. The period of time may be, for example, less than about 10 minutes. As further examples, the period of time may be about 30 minutes, about 1 hour, about 2 hours, about 6 hours, about 12 hours, about 24 hours, about 3 days, about 1 week, about 4 weeks, about 1 year, or more. Of course, the period of time is not limited to the values recited herein, but may be any period of time that is deemed suitable for a particular application.

The compositions disclosed herein may be capable of regulating or maintaining the concentration of a chemical target in a physiological fluid or ecosystem substantially within a predetermined range. The predetermined range, as described above, is generally the beneficial range of concentrations. Organisms are often, however, capable of surviving for extended periods of time even when the concentration of a component of the organisms' physiological fluid falls below the minimum beneficial concentration or above the maximum beneficial concentration. Therefore, the predetermined range may include values that are 20%, 30%, 40%, 50%, 60%, 70%, 80%, 90%, 95%, or 99% of the minimum value of the beneficial range of concentrations. The predetermined range may also include values that are 101%, 105%, 110%, 120%, 130%, 140%, 150%, 200%, 250%, 300%, 400%, or 500% of the maximum value of the beneficial range of concentrations.

The chemical target may be, for example, a metabolite which has a nominal concentration in the physiological fluid of an organism. The nominal concentration may be a range or a specific value. Nominal concentrations for metabolites in physiological fluids of humans, for example, vary with a number of factors including the age, sex, and weight of the human. However, these values are generally well known in the art. In one embodiment, then, the concentration of a metabolite in a human patient's blood prior to administration of a pharmaceutical composition according to the current disclosure may be between about 0.1 and about 100 times, or about 0.2 and about 10 times, or between about 0.5 and about 5 times, or between about 0.8 and about 3 times, or between about 0.9 and about 2 times, or between about 0.95 and about 1.5 times the nominal concentration in the blood for that patient. In addition, the concentration of the metabolite in the pharmaceutical composition may also be within these limits. Preferably, the binding constant of the binding moieties in the composition will be complementary with the nominal concentration of the metabolite for the patient. Such a binding constant will allow the pharmaceutical composition to modify (i.e., increase or decrease) the concentration of the metabolite in the patients' blood until the concentration reaches the nominal level.

In one method of use of the compositions described herein, the concentration of a chemical target in a physiological fluid of a patient is regulated. The method comprises contacting the physiological fluid with the composition of the invention. The contacting may occur within the body (in vivo), such as for an injectable or implantable composition, or outside the body (ex vivo), such as in a dialysis procedure. For example, the concentration of glucose in a patients' blood may be regulated using the compositions disclosed herein. The patient may be suffering from diabetes, for example. Such treatment may be effective in reducing glycemic variations and/or oxidative stress in the patient. For implants and other localized methods of treatment, the concentration of the chemical target may be regulated in either the tissue that is local to the implant, if an implant is used, or the concentration may be regulated systemically. The composition to be administered to the patient may or may not contain the chemical target. Furthermore, the chemical target may be administered separately to the patient, such as by injection. Further details of an example—i.e., a method for regulating glucose concentrations—are provided below.

In another method of use, the compositions described herein may be used to aid the normal functioning of a liver in an organism such as a human. The liver is able to remove toxins from the blood, although the rate of toxin removal is limited. In some cases, a toxin damages or kills an organism simply because the liver is not able to lower the concentration of a toxin in the blood quickly enough to prevent such damage. The compositions described herein may be useful as a supplemental reservoir for toxins in the blood. The composition binds with the toxin to lower the concentration of the toxin in the blood below toxic levels. As the liver removes unbound toxin from the blood, the composition continually releases bound toxin. The concentration of the toxin is maintained below toxic levels, and the liver is given sufficient time to process all of the toxin from the blood. This method may be useful, for example, in mitigating the risk to humans of certain fish and meat toxins.

In another method of use of the compositions described herein, the concentration of a chemical target in an aqueous environment of an ecosystem is regulated. The method comprises contacting the aqueous solution found in the ecosystem with the composition of the invention. The contacting may or may not occur within the aqueous environment itself. For example, in the case of a lake, the composition may be added to the lake, or the water may be removed from the lake, contacted with the composition, and returned to the lake. The composition to be administered to the ecosystem may or may not contain the chemical target. Furthermore, the chemical target may be administered separately to the ecosystem.

Product kits comprising the compositions disclosed herein as well as, for example, components for application of the composition and instruction guides, are also within the scope of the present invention.

### EXAMPLE

### Glucose Regulation

The following discussion describes one embodiment of the invention—a binding composition for regulating the concentration of glucose in the blood of a human—and is provided for the sake of illustration. It will be appreciated that the invention is in no way intended to be limited by this example, and furthermore that one of ordinary skill would be capable of applying the teachings presented herein to a wide range of binding compositions for regulating the concentration of chemical targets in physiological fluids and/or ecosystems.

The glucose-binding composition comprises a plurality of glucose binding moieties. The binding constant of the glucose binding moieties preferentially has a glucose-binding constant value complementary with the range of glucose concentration variations in a living body or ecosystem. The glucose-binding composition therefore binds glucose when glucose is elevated and dissociates glucose when glucose levels subside below normal levels. When the appropriate quantity of glucose-binding composition is implanted or placed in contact with the living body such that a rapid glucose exchange between blood glucose and glucose-binding implant can be take place, said implant reduces glycemic variations and the magnitude and duration of hyper- and hypo-glycemic events associated with diabetes.

The terms “glucose-binding composition” refers to a composition comprised of glucose binding moieties capable of reversibly binding glucose in vivo. Typically, the glucose-binding composition is capable of such reversible binding where the glucose concentrations in the physiological fluid (e.g., blood) is between 0.1 mM and 50 mM. Specifically, the binding constant of the binding moieties allows the binding moieties to be in equilibrium with the physiological fluid when the concentration of glucose in the physiological fluid is in the range of about 0.1 mM to about 50 mM.

General examples of glucose-binding moieties include boron-containing moieties such as boronic acid and boronate ion, arsenious acid, arsenite ion, telluric acid, tellurate ion, germanic acid, germanate ion, glucoseoxidase and glucose-binding moieties prepared by template or molecular-imprint polymerization. Each type of glucose-binding moiety has an associated binding constant. By incorporating varying amounts of one or another glucose-binding moiety within the binding component of the pharmaceutical composition, the overall binding capacity and equilibrium concentration of the composition may be varied.

A glucose-binding composition can be formulated in the form of small hydrogel particles allowing for rapid glucose diffusion and binding. Such implanted glucose binding composition serves as a “glucose buffer”. It binds glucose during hypoglycemic events and releases free glucose later as levels subside to normal or below normal. Since a certain amount of glucose is bound to the glucose-binding composition at normal glucose levels, the composition serves as a glucose source during hypoglycemic events by dissociating bound glucose at lower than normal levels. The amount of glucose bound to the composition at normal glucose levels and, respectively, its capacity to serve as a source of glucose in hypoglycemic events can be optimized by designing the glucose-binding composition with a higher or lower glucose binding constant.

In one aspect the glucose-binding composition comprises glucose-binding moieties linked to a polymeric matrix forming a hydrogel. Such hydrogel material is formed into particles ranging in diameter from 0.1 micrometers to 3 mm. The hydrogel matrix is biocompatible, non-immunogenic and optionally bioresorbable. Examples of suitable hydrogel material include polyethyleneglycol hydrogels, hyaluronic acid hydrogels and hydroxyethylmethacrylate hydrogels. The particles of hydrogel can be coated or covalently modified to reduce cell adhesion and provide a molecular weight cut-off function for diffusion into the hydrogel particles. One example of such a coating is the layer-by-layer polyelectrolyte method well described in literature (H. AI, J. Gao, Journal of Materials Science 39 (2004) 1429-1432). Polymer pairs that can be used for layer-by-layer coating of glucose binding compositions include sodium cellulose and polydiallyldimethylammonium chloride; polylysine and alginate; dextran sulfate and protamine; polystyrene sulfate and polydiallyldimethylammonium chloride.

In another aspect of this example a glucose binding composition is a biodegradable composition with a half-degradation time of a few days to several weeks or months. The breakdown products are biocompatible, non-immunogenic, non-toxic or have acceptable toxicity, are smaller then 20 kDa in molecular weight and are preferentially excreted via kidneys.

In another aspect of this example a glucose binding composition is used to modulate glucose concentration at the implant tissue site. In this embodiment the binding constant of the implant for glucose is adjusted such that at equilibrium the concentration of free glucose in proximity to the implant or inside of the implant is higher than the glucose concentration at the tissue site without implant. Such locally elevated glucose concentration can be beneficial for tissue regeneration and wound healing. Indeed, in one aspect of this example the glucose-binding composition can be placed into a wound, laceration, surgical wound, ulcer, or any other site undergoing repair and healing to promote, accelerate or enhance biological processes associated with tissue healing and regeneration. It has been shown in vitro studies that elevated extracellular glucose increased migration, adhesion and proliferation of human corneal epithelial (HCE) cells.

In one aspect of this example the glucose-binding composition can be used to treat diabetes. Functioning, in a sense, as an “artificial pancreas,” the composition may be formulated and introduced into the body (e.g., in the form of an implant or solution) such that the composition regulates the concentration of glucose in the blood of a diabetic patient. Thus, the equilibrium glucose concentration of the composition is chosen to approximate the “normal” equilibrium concentration in the blood of the patient. Hypoglycemia and hyperglycemia are regulated by the release and/or uptake of glucose by the glucose-binding composition.

In another aspect of this example the glucose-binding composition can be used in conjunction with other therapies used treat diabetes, including various insulin therapies listed below: NovoLog (Novo Nordisk), Iletin I Regular (Lilly), Humulin R (Lilly), Novolin R (Novo Nordisk), Velosulin BR (Lilly), NPH Iletin II (Lilly), Humulin N (Lilly), Novolin N (Novo Nordisk), Lente Iletin II (Lilly), Humulin L (Lilly), Novolin L (Novo Nordisk), Humulin U Ultralente (Lilly), Humulin 70/30 (Lilly), Lantus (Aventis), Novolin 70/30 (Novo Nordisk), Humulin 50/50 (Lilly), Humalog Mix 75/25 (Lilly), NovoLog Mix 70/30, and oral pharmaceuticals including glyburide (Diabeta®, Micrometersase®, Glynase®), glipizide (Glucotrol®, Glucotrol XL®), glimepiride (Amaryl®), repaglinide (Prandin®), nateglinide (Starlix®), metformin hydrochloride (Glucophage®), pioglitazone hydrochloride (Actos®), rosiglitazone malate (Avandia®), acarbose (Precose®, Glucobay®) and miglitol (Glyset®). For example, since the glucose-binding implant has the capacity to bind glucose at hyperglycemic levels, thus normalizing glucose levels, insulin and other medications can be used less frequently. In cases of persistent hyperglycemia, the capacity of the glucose-binding implant may be exceeded. An injection of insulin or intake of oral diabetes drugs typically reduces the concentration of glucose in blood and this will cause dissociation of the bound glucose from the glucose-binding implant. Thus the capacity of glucose-binding implant can be “recycled”. The glucose released from the glucose-binding implant after insulin injection may be beneficial in avoiding medication induced hypoglycemia.

In one aspect of this example, boron-containing compounds are used to prepare the glucose-binding component of the composition. It is known that boronic acids form cyclic esters with saccharides and the reaction occurs reversibly and rapidly at ambient temperature. It has been demonstrated that boronic acids serve as a useful interface to selectively recognize saccharides in water.

Other examples of boronate moieties and compounds suitable for reversible binding of glucose are phenylboronic acid, 2-carboxyethaneboronic acid, 1,2-dicarboxyethaneboronic acid, β,β′-dicarboxyethaneboronate, β,γ-dicarboxypropaneboronate, 2-nitro- and 4-nitro-3-succinamidobenzene boronic acids, 3-nitro-4-(6-aminohexylamido)-phenyl boronic acid, {4-[(hexamethylenetetramine)methyl]phenyl}boronic acid, 4-(N-methyl)carboxamidobenzene boronic acid, 2-{[(4-boronphenyl)methyl]-ethylammonio}ethyl and compounds containing 2-{[(4-boronphenyl)methyl]diethylammonio}ethyl groups, succinyl-3-aminophenylboronic acid, 6-aminocaproyl-3-aminophenylboronic acid, 3-(N-succinimidoxycarbonyl)aminophenylboronate, p-(omega-aminoethyl)phenylboronate, p-vinylbenzeneboronate, N-(3-dihydroxyborylphenyl)succinamic acid, N-(4-nitro-3-dihydroxyborylphenyl)succinamic acid, O-dimethylaminomethylbenzeneboronic acid, 4-carboxybenzeneboronic acid, 4-(N-octyl)carboxamidobenzeneboronic acid, 3-nitro-4-carboxybenzeneboronic acid, 2-nitro-4-carboxybenzeneboronic acid, 4-bromophenylboronate, p-vinylbenzene boronate, 4-(.omega.-aminoethyl)phenylboronate, catechol[2-(diethylamino)carbonyl, 4-bromomethyl]phenyl boronate, and 5-vinyl-2-dimethylaminomethylbenzeneboronic acid and boronic moieties described in U.S. Pat. Nos. 6,927,246 and 6,858,592 and incorporated herein by reference. Further examples of glucose binding moieties include those described in U.S. Pat. No. 6,916,660, which is also incorporated by the reference.

In one embodiment of this example, the glucose-binding moieties of the invention may be immobilized in a saccharide-permeable biocompatible polymer matrix to form an implantable “glucose buffer”. Suitable biocompatible polymer matrices used for medical implants are known in the art. The glucose-binding moieties can be covalently bound to the polymer matrix using techniques such as those described in U.S. Pat. No. 6,002,954, which is hereby incorporated by reference. Such methods generally involve adding a suitable tether to the molecule such that the tether can be used to covalently attach the compound to the matrix.

The aryl boronic acid compounds of the present invention can also be reacted to form boronate esters with polymers having free alcohol or diol groups. Reactions for forming boronate ester bonds are well known in the art and include refluxing the boronic acid and diol in an appropriate solvent (e.g., alcohol, toluene, methylene chloride, tetrahydrofuran or dimethyl sulfoxide). Alternatively, an aryl boronic acid can be added to a polymer having free alcohol or diol groups by means of a transesterification reaction, as described in D. H. Kinder and M. M. Ames, Journal of Organic Chemistry 52:2452 (1987) and D. S. Matteson and R. Ray, Journal of American Chemical Society 102:7590 (1980), the entire teachings of which are incorporated herein by reference.

The complexation of carbohydrates, including glucose, with phenylboronic acid has been known for a long time and the reversibility of that interaction has served as a basis for the chromatographic separation of sugars. Specifically, in 1959, Lorand and Edwards reported association constants for aqueous associations of phenylboronic acid with many saturated polyols; binding interactions ranged from very weak (e.g., ethylene glycol, K_{d}=360 mM) to moderately strong (e.g., glucose, K_{d}=9.1 mM). See J. Yoon, et al., Bioorganic and Medicinal Chemistry 1(4):267-71 (1993). The binding mechanism is believed to occur through bonding of adjacent hydroxyl groups on glucose to hydroxyl groups on a boronate moiety. Other references describing interaction of glucose with boronic acid and synthesis of boronate containing polymers include James et al. “A saccharide ‘sponge’ Synthesis and Properties of a dendritic boronic acid “Chemical Communications 6:705-706 (1996); Kimura et al., “Sugar-induced conformational changes in boronic acid-appended poly(L- and D-lysine)s and sugar-controlled orientation of a cyanine dye on the polymers” Journal of the Chemical Society Perkin Transaction 2 10:1884-1894 (1995); Liu et al., “New Ligands for boronate affinity chromatography” Journal of Chromatography A 687:61-69 (1994); Malan et al., “Synthesis of 4-Borono-L-phenylalanine” Synlett 2:167-168 (1996); Miyazaki et al., “Boronate-Containing Polymer as Novel Mitogen for Lymphocytes” Biochemical and Biophysical Research Communications 195:829-836 (1993); Shiino, et al. “Preparation and characterization of a glucose-responsive insulin-releasing polymer device” Biomaterials 15(2): 121-128 (1994); Shiino, et al. “A Self-Regulated Insulin Delivery System Using Boronic Acid Gel” Journal of Intelligent Material Systems and Structures 5:311-314 (1994); Shiino et al., “Amine effect on phenylboronic acid complex with glucose under physiological pH in aqueous solution” Journal of Biomaterials Science Polymer Edition 7:697-705 (1996); Singhal, et al. “New Ligands for boronate affinity chromatography: Synthesis and properties” Journal of Chromatography 543:17-38 (1991) and in U.S. Pat. No. 6,927,246.

Often, compounds which interact with glucose in the manner described above also have a tendency to interact with other compounds having hydroxyl groups, thus reducing the specificity of a glucose assay, especially when assaying physiological samples which may contain interfering amounts of lactate or acetoacetate, among other compounds. For example, some diabetic patients also develop lactic acidosis, in which blood lactate levels are greater than 5 mmol/liter. A glucose-binding implant insensitive to potentially interfering hydroxyl compounds, such as lactate, will have higher capacity for minimizing glycemic variations in diabetic patients.

In one aspect, the present invention provides a way to preferentially bind glucose in a metabolic condition which may result in the presence of interfering compounds, such as alpha-hydroxy acids or beta-diketones. Such potentially interfering compounds include lactate, acetoacetate, beta-hydroxy butyric acid, and the like. In one embodiment of the example, a glucose-binding composition is used which is capable of binding glucose from the physiological fluid, but which is less likely to bind interfering compounds in the physiological fluid. The glucose-binding composition has at least two recognition elements for glucose, oriented such that the interaction between the composition and glucose is more stable than the interaction between the composition and the interfering compounds. Increase in selectivity using two site recognition of glucose is described in Arimori S. Sugar, “Sensing by Chiral Orientation of Dimeric Boronic Acid Appended Porphyrins Which Show Selectivity for Glucose and Xylose.” Chemistry Letters 1996 vol. 1, 77-78., and in U.S. Pat. No. 6,800,451.

The multi-site recognition and binding of glucose on or within a support immobilized glucose recognition element can be accomplished by spatial arrangement and density of glucose recognition elements in the glucose-binding implant matrix.

Suitable recognition elements for multi-recognition include moieties which are capable of a preferably reversible interaction with glucose, especially with the diol groups present in glucose. Several such recognition elements are known, and preferably include compounds containing boron such as boronic acid and boronate ion, arsenious acid, arsenite ion, telluric acid, tellurate ion, germanic acid, germanate ion, and the like. The recognition elements are spaced on the glucose binding composition a suitable distance from each other so as to allow at least two of the recognition elements to interact with a glucose molecule, resulting in increased specificity. In general, the recognition elements may have a spacer of up to about 30 atoms between them. Preferably, the recognition elements are oriented such that they are capable of being about 6 Angstroms apart when interacting with glucose.

In one embodiment the glucose-binding formulation is placed into the peritoneal cavity in the form of particles, beads, sheets, strings, hydrogel, solution, bag, or capsules filled with glucose-binding compositions for a period of time ranging from 5 min to 24 hours, or from 24 hours to several days, or from several days to several weeks, or from several weeks to several months, or from several months to several years, and thereafter removed surgically or via an access port by aspiration, morcelation, or other acceptable methods. Alternatively, the formulation may be prepared using a bioerodible composition, precluding the need to surgically remove the formulation. The formulation may bioerode over a period of hours, days, months, or years, as appropriate.

In one aspect of this invention a glucose-binding composition is implanted in peritoneal cavity in the amount sufficient to bind glucose several fold exceeding glucose amounts present in circulating blood. Elevated glucose levels are typically found in diabetic patients after ingestion of food and, if insulin therapy is not administered, can persist for several hours until excess of blood glucose is metabolized by the body. The peritoneal surface is known for rapid exchange of blood metabolites during peritoneal dialysis.

In yet another aspect of this example a water soluble polymer, having molecular weight 30,000-10,000,000 Da, or an extrudable hydrogel, or a crosslinked polymer with covalently bound glucose-binding moieties is used with a peritoneal glucose dialysis device to manage glucose levels in diabetic patients. Examples of such polymers include hyaluronic acid, crosslinked hyaluronic acid (X. Shu, Y. Liu, Y. Luo, M. Roberts, G. Prestwich, Biomacromolecules, 3: 1304-1311 (2002)), polyethylenglycol hydrogels, high molecular weight dextrans, high molecular weight extrudable crosslinked polyethylenglycol polymers, thermoreversible polymers all containing covalently bound glucose-binding moieties such as phenylboronic acid derivatives. This approach is particularly useful for managing glycemic levels in hospitalized patients with diabetes.

The peritoneal glucose dialysis device in this embodiment may have only the dialysis part placed into the peritoneal cavity or the whole device may be implanted into the peritoneal cavity. The dialysis device is comprised of a reservoir for the glucose-binding polymer, a dialysis part comprised of a semi-permeable membrane with a molecular weight cut-off value sufficient to prevent the glucose-binding polymer from diffusing into the peritoneal cavity, a recirculation pump, and optionally a glucose sensor to monitor levels of glucose bound to the glucose-binding implant. The dialysis part of the device may have the shape of a tube and be inserted into the peritoneal cavity via throat or other surgical port. Similar to existing implantable insulin pumps, the reservoir may be accessed via an injection port to replace the glucose-binding polymer.

The glucose dialysis device described above can be used as a part of an “artificial pancreas” unit comprising peripheral blood glucose sensor, insulin and/or other diabetes medication pump, glucose infusion pump, other medication delivery devices and a computer control unit.

### EXPERIMENTAL

Unless otherwise indicated, the practice of the present invention will employ conventional techniques of, for example, organic chemistry, polymer chemistry, medicinal chemistry, and pharmaceutical formulation, which are within the skill of the art. Such techniques are explained fully in the literature.

### Example 1

### A Glucose-Modulating Resorbable Microparticular Implant

Hydrogel particles ranging in diameter between 100 and 200 micrometers are prepared by emulsion polymerization of or a 20% (w/w) aqueous solution at pH 8.0 of hydroxyethylmethacrylate-boronate (HEMA-boronate) and 4 armed PEG-acrylate having molecular weight between 8,000 and 10,000 Da. Toluene or another non-water miscible solvent that also does not dissolve the HEMA-boronate or PEG-acrylate is used to form the emulsion. TEMED and persulfate are used as reaction initiators. The HEMA-boronate reagent is synthesized by forming a covalent link between hydroxyl groups of HEMA and reactive groups on a phenylboronate moiety. Reactions for forming boronate ester bonds are well known in the art and include refluxing the boronic acid and diol in an appropriate solvent (e.g., alcohol, toluene, methylene chloride, tetrahydrofuran (THF) or dimethyl sulfoxide (DMSO)). Alternatively, an aryl boronic acid can be added to a polymer having free alcohol or diol groups by means of a transesterification reaction, as described (D. H. Kinder and M. M. Ames, Journal of Organic Chemistry 52:2452 (1987) and D. S. Matteson and R. Ray, Journal of American Chemical Society 102:7590 (1980), Boronic Acids. Edited by Dennis G. Hall (2005) WILEY-VCH Verlag GmbH & Co. KGaA, Weinheim, ISBN 3-527-30991-8), the entire teachings of which are incorporated herein by reference. The PEG-Acrylate reagent is prepared by reacting acryloyl-chloride with polyethylenglycol polymer having pentaerythritol (2,2-bis(hydroxymethyl)-1,3-propanediol) core and molecular weight of approximately 10,000 Da. The biodegradation rate of PEG/HEMA/Boronate particles can be modulated by introduction of thioester bonds in the structure of the particle polymer. The polymer can be prepared to have a half-biodegradation time in vivo from a few days to several months. Washed and equilibrated in physiological solution, particles are introduced into the peritoneal cavity in the form of an injectable or flowable suspension. Since implanted particles typically are bioresorbed in several weeks, the foreign body reaction to the implant is minimal. The ongoing hydrolysis of polymer and boronate-containing groups from the surface of the particles prevents the fouling of the particle surface by glycoproteins. Resorbable glucose-modulating boronate implants are useful for managing glycemic variation in trauma patients, patients in hospital intensive care units, women with pregnancy-induced diabetes and in other conditions causing acute and transient dysglycemia.

### Example 2

### Long-Term Glucose-Modulating Implant

Polymethacrylic acid particles are prepared by emulsion polymerization to yield hydrogel particles with nominal size 200 micrometers. The particles are covalently modified with a phenylboronic acid derivative, and thereafter encapsulated into polyelectrolyte multilayers using layer-by-layer polymer adsorption as described in published US Patent Applications Nos. 2005/0196520, 2005/0191430, 2004/0063200. Recently, polyelectrolyte multilayers (PEM) have been studied as bioinert films to reduce cell and protein adhesion (Elbert, D. L. et al. [1999] Langmuir 15:5355-5362) and as coatings to modulate interfacial molecular transport in drug delivery and immunoisolation systems (Moya, S. et al. [2000] Macromolecules 33:4538-4544; Shi, X. and -Caruso, F. [2001] Langmuir 17:2036-2042). Coating or encapsulation of boronate-polymer hydrogel particles provides an effective molecular weight cut-off, of approximately 3,000 Da, for diffusion in and out of the hydrogel particles. Such molecular weight cut-off layer is important for retaining the long-term functionality of the implant because it prevents diffusion and binding of glycoproteins to the boronate-hydrogel. To establish a close interface and rapid exchange between the chemical target in peripheral blood and the implant particles, the particles are further encapsulated in a double layered e-PTFE material (from WL Gore & Associates, Inc.). The first layer of this composite material is an e-PTFE layer with porosity of 5-20 micrometers, and the second layer is the bioisolation layer of with pore diameter of 1-2 micrometers. As was described above, the angiogenic layer promotes ingrowth of microvasculature into the pores of e-PTFE. The new microvasculature is established inside the e-PTFE material within two to three weeks after implantation. The glucose-binding implant is expected to retain at least 80% of its functionality in vivo for several months.

### Example 3

### Synthesis of Glucose-Binding Implant (GBI) Using 4-Vinylphenylboronic Acid

Four mole-equivalents of 4-vinylphenylboronic acid (available from Fisher Scientific) are copolymerized by radical polymerization with one mole-equivalent of PEG-Acrylate reagent prepared by reacting acryloyl-chloride with polyethylenglycol polymer having pentaerythritol (2,2-bis(hydroxymethyl)-1,3-propanediol) core and molecular weight of approximately 3,000 Da. The synthesis of a GBI hydrogel containing 20% of PEG-boronate can be performed at a reasonably low temperature (+4° C.) in aqueous system by using potassium persulfate and tetramethylenediamine as the redox system.

### Example 4

### Determination of the Required Glucose-Binding Capacity, Volume of the Glucose-Binding Implant and Toxicity of Implant Breakdown Products

In order to make glucose-binding implant (GBI) to effectively modulate glucose concentration in human body the capacity of such implant has to be sufficient to bind excessive glucose in blood of hyperglycemic patients. In severe hyperglycemia glucose levels may reach 600 mg/dl. Assuming average volume of blood in a human being is 5 liters and normal range of glucose in blood is approximately 100 mg/dl the glucose binding implant has to be able to bind 25 grams or 0.14 moles (600 mg/dl−100 mg/dl)×50 dl=25 grams) of glucose. When GBI is a hydrophilic polymer such as polyethylenglycol and glucose-binding moieties immobilized statistically one per every 20 PEG backbone monomer repeats to immobilize 0.14 moles of glucose will require 123 grams of PEG polymer (0.14Mo×20 units of PEG x 44 (mol. weight)=123 grams). Assuming the GBI is a PEG hydrogel containing 20% of PEG-phenylboronate the volume of the GBI hydrogel that will be required for introduction into the human body will be approximately 615 ml. An implant of such volume is considered to be acceptable for the implantation into peritoneal cavity. Depending on the polymerization and crosslinking chemistry used in the synthesis of the GBI the implant may be resorbed in several weeks or months after implantation. Chronic intravenous infusion of 90 mg/kg/day of PEG with molecular weight of 3,350 Da for 178 days showed no adverse effects in dogs (Working, P. K. et al. 1997). Experiments with chronic exposure to boric acid showed no observed adverse effect at 9-43 mg boron/kg/day (Hubbard, S. A. 1998). Assuming 30 days for half degradation of the implant in the peritoneal cavity and average human body weight of 70 kg the levels of chronic exposure to boronate (half of 0.14 moles×45 (MW of boronate moiety) over 30 days and 70 kg=1.5 mg/kg/day) and to PEG degradation fragments (which assumed to have MW 3,000 Da; half of 123 g over 30 days and 70 kg of body weight=29 mg/kg/day) are significantly below the safe reported levels for boronate and PEG degradation products.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains.

Throughout the specification various patents, patent applications and other publications are referenced. The entire content of these patents, patent applications and other publications are incorporated herein by reference. Express definitions in any of the references cited herein apply only to the reference in which they are provided.

What is claimed is:

1. A pharmaceutical composition comprising a biocompatible binding component comprising a binding moiety capable of reversibly binding with a chemical target, wherein the pharmaceutical composition is capable of regulating the concentration of the chemical target in a physiological fluid of a patient.

2. The pharmaceutical composition of claim 1, further comprising the chemical target.

3. The pharmaceutical composition of claim 1, wherein the binding constant of the binding moiety is complementary to the beneficial range of concentrations of the chemical target in the physiological fluid.

4. The pharmaceutical composition of claim 1, wherein the pharmaceutical composition is capable of maintaining the concentration of the chemical target substantially within a predetermined range for a predetermined period of time.

5. The pharmaceutical composition of claim 1, wherein the pharmaceutical composition is suitable for administration to a patient.

6. The pharmaceutical composition of claim 5, wherein the pharmaceutical composition is suitable to be administered to a patient parenterally, orally, rectally, vaginally, sublingually, nasally, topically, or transdermally.

7. The pharmaceutical composition of claim 5, wherein the pharmaceutical composition is suitable to be administered to a patient via implantation.

8. The pharmaceutical composition of claim 1, further comprising a pharmaceutically acceptable carrier.

9. The pharmaceutical composition of claim 8, wherein the pharmaceutically acceptable carrier is a polymer matrix.

10. The pharmaceutical composition of claim 8, wherein the pharmaceutically acceptable carrier is a hydrogel.

11. The pharmaceutical composition of claim 8, wherein the binding component is dispersed within the pharmaceutically acceptable carrier.

12. The pharmaceutical composition of claim 10, wherein the binding moieties are covalently attached to the hydrogel.

13. The pharmaceutical composition of claim 4, wherein the predetermined range is the beneficial range of concentrations for the chemical target in the physiological fluid.

14. The pharmaceutical composition of claim 4, wherein the predetermined range includes concentrations that are about 20% or more of the minimum beneficial concentration for the chemical target in the physiological fluid.

15. The pharmaceutical composition of claim 4, wherein the predetermined range includes concentrations that are about 500% or less of the maximum beneficial concentration for the chemical target in the physiological fluid.

16. The pharmaceutical composition of claim 4, wherein the predetermined period of time is about 1 hr.

17. The pharmaceutical composition of claim 4, wherein the predetermined period of time is about 24 hr.

18. The pharmaceutical composition of claim 4, wherein the predetermined period of time is about 1 week.

19. The pharmaceutical composition of claim 4, wherein the predetermined period of time is about 4 weeks.

20. The pharmaceutical composition of claim 1, wherein the pharmaceutical composition is in the form of a solution.

21. The pharmaceutical composition of claim 1, wherein the pharmaceutical composition is in the form of a hydrogel, hydrogel particles, hydrogel beads, or a hydrogel paste.

22. The pharmaceutical composition of claim 1, wherein the binding component comprises a polymer.

23. The pharmaceutical composition of claim 1, wherein the binding component comprises an elastomer.

24. The pharmaceutical composition of claim 1, wherein the binding component comprises a plurality of polymers that form a hydrogel upon mixing.

25. The pharmaceutical composition of claim 5, wherein the binding component is bioresorbable.

26. The pharmaceutical composition of claim 2, wherein the chemical target is selected from nutrients, hormones, drugs, toxins, cells, vitamins, antibodies, proteins, nucleic acids, electrolytes, and enzymes.

27. The pharmaceutical composition of claim 2, wherein the chemical target is a nutrient.

28. The pharmaceutical composition of claim 27, wherein the chemical target is glucose.

29. The pharmaceutical composition of claim 28, wherein the binding component comprises a plurality of glucose binding moieties.

30. The pharmaceutical composition of claim 29, wherein the binding constant of the binding moieties is such that the binding moieties to be in equilibrium with the physiological fluid when the concentration of glucose in the physiological fluid is in the range of about 0.1 mM to about 50 mM.

31. The pharmaceutical composition of claim 30, wherein the binding component comprises boronic acid, boronate ion, arsenious acid, arsenite ion, telluric acid, tellurate ion, germanic acid, germanate ion, or analogs or derivatives thereof.

32. The pharmaceutical composition of claim 31, wherein the binding component comprises phenylboronic acid or analogs or derivatives thereof.

33. The pharmaceutical composition of claim 28, wherein the binding component preferentially binds glucose in vivo over metabolites present in physiological fluids containing alpha-hydroxy or beta-diketone groups.

34. The pharmaceutical composition of claim 30, wherein the glucose binding moiety is covalently attached to a polymeric matrix.

35. The pharmaceutical composition of claim 34, wherein the glucose binding moiety is phenylboronic acid or derivatives thereof.

36. The pharmaceutical composition of claim 2, wherein the chemical target is a metabolite and the concentration of the metabolite in the pharmaceutical composition is between about 0.2 and 10 times the nominal concentration of the metabolite in the physiological fluid.

37. The pharmaceutical composition of claim 2, wherein the chemical target is selected from endogenous autologous hormones, endogenous autologous cytokines, thyroid stimulating hormones, toxins, herbicides, pesticides, fertilizers, chemical warfare agents, environmental pollutants, heavy metals, viruses, prions, and plasmids.

38. The pharmaceutical composition of claim 2, wherein the chemical target is selected from endogenous PDGF, FGF, bone morphogenic protein, EGF, TGF-β, carbon monoxide, triiodothyronine, nitric oxide, thyroxine, and autologous or synthetic insulin.

39. The pharmaceutical composition of claim 7, further comprising angiogenic material selected from hydrophilic polyvinylidene fluoride, mixed cellulose esters, e-PTFE, polyester, polyvinyl chloride, polypropylene, polyethylene, polysulfone, polyethersulfone, cellulose acetate, nylon, polycarbonate, polymethylmethacrylate, and mixtures thereof.

40. The pharmaceutical composition of claim 39, further comprising a layer of bioprotective material.

41. The pharmaceutical composition of claim 40, wherein the bioprotective material comprises a material selected from polyurethane, polytetrafluoroethylene, polypropylene, polyethylene, and polysulfone.

42. The pharmaceutical composition of claim 40, wherein the layer of bioprotective material is located between the binding component and the angiogenic material.

43. The pharmaceutical composition of claim 40, wherein the angiogenic material and the bioprotective layer together comprise a composite membrane.

44. A pharmaceutical composition for treating diabetes comprising a biocompatible binding component capable of reversibly binding glucose, wherein the pharmaceutical composition is in a dosage form capable of contacting the physiological fluid of a patient suffering from diabetes for a predetermined length of time.

45. The pharmaceutical composition of claim 44, wherein the pharmaceutical composition is in the form of a bolus.

46. The pharmaceutical composition of claim 44, further comprising glucose.

47. A method of regulating the concentration of a chemical target in a physiological fluid of a patient, comprising contacting the physiological fluid with the composition of claim 1.

48. A method of lowering the concentration of a chemical target in a physiological fluid in a patient, comprising contacting the physiological fluid with the composition of claim 1.

49. A method of increasing the concentration of a chemical target in a physiological fluid in a patient, comprising contacting the physiological fluid with the composition of claim 1.

50. The method of claim 47, wherein the composition is maintained outside of the body of the patient during the contacting.

51. The method of claim 47, wherein the composition is administered to the patient and the contacting occurs in vivo.

52. The method of claim 51, wherein the composition is administered parenterally, orally, rectally, vaginally, sublingually, nasally, topically, or transdermally.

53. The method of claim 51, wherein the composition is implanted into the patient.

54. A method for regulating the concentration of glucose in the blood of a patient comprising contacting the blood of the patient with a pharmaceutical composition comprising a therapeutic amount of a biocompatible binding component capable of reversibly binding glucose.

55. The method of claim 54, wherein the patient suffers from diabetes.

56. The method of claim 54, wherein the method is effective for reducing glycemic variations in the patient.

57. The method of claim 54, wherein the method is effective for reducing oxidative stress in the patient.

58. The method of claim 54, wherein the pharmaceutical composition is capable of the reversible binding of glucose when the physiological fluid has a glucose concentration within the range of 0.1 mM to 50 mM.

59. The method of claim 54, wherein the pharmaceutical composition is implanted in the body of the patient, and the contacting occurs in vivo.

60. The method of claim 59, wherein the pharmaceutical composition is implanted into muscle tissue, subcutaneous tissue, bone, liver tissue, the peritoneal cavity, the thoracic cavity, a blood vessel, lung tissue, brain tissue, the cerebrospinal canal, eye tissue, kidney tissue, spleen tissue, fatty tissue, or bladder tissue.

61. The method of claim 59, wherein the pharmaceutical composition regulates the concentration of glucose in the local tissue.

62. The method of claim 59, wherein the pharmaceutical composition regulates the systemic concentration of glucose.

63. The method of claim 54, wherein the contacting occurs outside of the body of the patient.

64. The method of claim 54, wherein the pharmaceutical composition further comprises glucose.

65. A method for regulating the concentration of a chemical target in a physiological fluid of a patient, the method comprising:
(a) administering to the patient a composition capable of binding to the chemical target; and
(b) administering to the patient the chemical target.

66. A composition for regulating the concentration of a chemical target in an aqueous environment of an ecosystem, comprising a binding component comprising a binding moiety capable of reversibly binding with a chemical target, wherein the binding constant of the binding moiety is complementary to the beneficial range of concentrations for the chemical target in the aqueous environment.

67. The composition of claim 66, wherein the composition is contained within a containment structure comprising a barrier that allows diffusion of the chemical target across the barrier.

68. The composition of claim 66, wherein the composition is capable of maintaining the concentration of the chemical target substantially within a predetermined range for a predetermined period of time.

69. The composition of claim 68, wherein the predetermined range the beneficial range of concentrations for the chemical target in the aqueous environment.

70. The composition of claim 68, wherein the predetermined range includes concentrations that are about 20% or more of the minimum beneficial concentration for the chemical target in the aqueous environment.

71. The composition of claim 68, wherein the predetermined range includes concentrations that are about 500% or less of the maximum beneficial concentration for the chemical target in the aqueous environment.

72. The composition of claim 66, wherein the composition is in the form of beads.

73. The composition of claim 66, wherein the composition is in the form of a hydrogel.

74. A method for regulating the concentration of a chemical target in an aqueous environment of an ecosystem, the method comprising delivering to the aqueous environment the composition of claim 66.
